(19) United States
(12) Patent Application Publication
(10) Pub. No.: US 2019/0119635 A1
(43) Pub. Date: Apr. 25, 2019

(54) MODULATION OF T LYMPHOCYTES

(71) Applicant: Fate Therapeutics, Inc., San Diego, CA (US)

(72) Inventors: David Robbins, Temecula, CA (US); Betsy Denise Rezner, San Diego, CA (US); Lisa Guerrettaz, San Diego, CA (US); Leah Mitchell, San Diego, CA (US)

(21) Appl. No.: 15/571,785

(22) PCT Filed: May 4, 2016
(86) PCT No.: PCT/US2016/030782

§ 371 Date: Nov. 3, 2017

Related U.S. Application Data
(60) Provisional application No. 62/157,160, filed on May 5, 2015.

(51) Int. Cl. C12N 5/0783; A61K 35/17

(52) U.S. Cl. CPC C12N 5/0636; C12N 5/0637; A61K 35/17; C12N 2510/00; C12N 2501/02; C12N 2501/999; C12N 2501/39

(57) ABSTRACT

The present disclosure relates to molecular biology, cell biology and immunology. Specifically, the present disclosure provides compositions and methods for modulating an isolated population of T lymphocytes to improve the therapeutic potential thereof.

### RELATED APPLICATION

This application claims priority to U.S. Provisional Application Ser. No. 62/157,160, filed May 5, 2015, the disclosures of which is hereby incorporated by reference in their entirety.

### BACKGROUND OF THE INVENTION

Cell based therapies have curative potential for a number of life threatening blood malignancies. Currently, their utility is limited by both low engraftment rate and a number of side-effects including a condition called graft versus host disease (“GvHD”). GvHD occurs when the immune cells from the donor, namely the T lymphocytes, identify that the host is not “self” and proceed to initiate an immune response against the recipient. GvHD can occur in highly matched donor/recipient transplants but is more common as HLA matching is more disparate. Several trials are underway that utilize T cell depletion to reduce the likelihood of GvHD when using a haploidentical (half match) or alternative low matching donors. Complete removal of T lymphocytes can prevent GvHD but also limits their roles in antitumor immune response or engraftment. Alternatively, large numbers of even “minor mismatched” T lymphocytes can result in high grade, life threatening GvHD and graft failure. Current T lymphocyte depletion approaches focus on only removing a fraction or subset of the T lymphocytes to balance the reduced GvHD with the benefits these cells have on antiviral and/or anti-tumor response.

Meanwhile, one challenge for T lymphocyte based cell therapy is in vivo expansion of T lymphocytes, rapid disappearance of the cells after infusion, and disappointing clinical activity (Jena, et al., Blood (2010), 1:16, 1035-1044; Uckun, et al, Blood, 1988, 71: 13-29). Thus, there is an urgent need in the art for compositions and methods for treatment of cancer using T lymphocytes that can have a sustained, therapeutic effect without the deleterious effects of rapid in vivo T lymphocyte expansion and concomitant GvHD and disappearance.

Therefore, identification of therapeutic applications of T lymphocytes that avoid GvHD and maintain a viable T lymphocyte population represent unmet needs. The methods and compositions of the present invention meet these needs and provide other related advantages.

### SUMMARY OF THE INVENTION

The present invention provides compositions and methods for modulating isolated populations or subpopulations of T lymphocytes to improve their therapeutic potential. In some embodiments, the present invention provides an isolated population or subpopulation of T lymphocytes that have been contacted ex vivo with an agent selected from a prostaglandin pathway agonist, a glucocorticoid, or a combination thereof. In some embodiments, the present invention provides a method of modulating an isolated population or subpopulation of T lymphocytes by contacting the isolated population or subpopulation of T lymphocytes ex vivo with an agent selected from a prostaglandin pathway agonist, a glucocorticoid, or a combination thereof.

In some embodiments, the isolated population of T lymphocytes can be peripheral blood mononuclear cells (PBMC), peripheral blood leukocytes (PBL), tumor infiltrating lymphocytes (TIL), or a combination thereof. In some embodiment, the isolated population of T lymphocytes can be selected from the group consisting of CD4+/CD8+ double positive T cells, cytotoxic T cells, Th3 (Treg) cells, Th9 cells, Thαβ helper cells, Tfh cells, stem memory TSCM cells, central memory TCM cells, effector memory TEM cells, effector memory TEMRA cells, gamma delta T cells and any combination thereof.

In some embodiments, the isolated subpopulation of T lymphocytes can have two or more types of T lymphocytes selected from the group consisting of CD4+/CD8+ double positive T cells, CD4+ T cells, CD8+ T cells, naive T cells, effector T cells, cytotoxic T cells, helper T cells, memory T cells, regulator T cells, Th0 cells, Th1 cells, Th2 cells, Th3 (Treg) cells, Th9 cells, Thαβ helper cells, Tfh cells, stem memory TSCM cells, central memory TCM cells, effector memory TEM cells, effector memory TEMRA cells, and gamma delta T cells.

In some embodiments, the isolated population of T lymphocytes can be differentiated in vitro from a stem cell, a definitive hemogenic endothelium, a CD34+ cell, a HSC (hematopoietic stem and progenitor cell), a hematopoietic multipotent progenitor cell, or a T cell progenitor cell. The stem cell can be an induced pluripotent stem cell (iPSC), or an embryonic stem cell (ESC).

In some embodiments, the isolated population of T lymphocytes can have an exogenous nucleic acid that encodes a T cell receptor (TCR) or a chimeric antigen receptor (CAR).

In some aspects, the T lymphocytes are human cells. In some aspects, T lymphocytes are CD3+ T lymphocytes. In some embodiments, the isolated population or subpopulation of T lymphocytes are obtained from peripheral blood, cord blood, or bone marrow.

In some embodiments, the prostaglandin pathway agonist can be a PGE receptor agonist. In some aspects, the PGE receptor agonist can be a compound that selectively binds the PGE2 EP2 or PGE2 EP4 receptor. In some embodiments, the prostaglandin pathway agonist can be PGE2, or a PGE2 derivative or analogue, such as PGE2, dmPGE2, 15(S)-15-methyl PGE2, 20-ethyl PGE2, or 8-iso-16-cyclohexyl-tetranor PGE2.

In some embodiments, the prostaglandin pathway agonist is selected from the group consisting of PGE2, prostaglandin 12, 16,16-dimethyl PGE2 (dmPGE2); 16,16-dimethyl PGE2 p-(p-acetamidobenzamido) phenyl ester; 11-deoxy-16,16-dimethyl PGE2; 9-deoxy-9-methylene-16,16-dimethyl PGE2; 9-deoxy-9-methylene PGE2; 9-keto Fluprostenol; 5-trans PGE2; 17-phenyl-omega-trinor PGE2; PGE2 serinol amide; PGE2 methyl ester; 16-phenyl tetranor PGE2; 15(S)-15-methyl PGE2; 15(R)-15-methyl PGE2; 8-iso-15-keto PGE2; 8-iso PGE2 isopropyl ester; 8-iso-16-cyclohexyltetranor PGE2; 20-hydroxy PGE2; 20-ethyl PGE2; 11-deoxy PGE1; nocloprost; sulprostone; butaprost; 15-keto PGE2; 19(R) hydroxy PGE2; 5-[(1E,3R)-4,4-difluoro-3-hydroxy-4-phenyl-1-buten-1-yl]-1-[6-(2H-tetrazol-5R-yl)hexyl]-2-pyrrolidinone (L-902,688); 2-[3-[(1R,2S,3R)-3-hydroxy-2-[(E,3S)-3-hydroxy-5-[2-(methoxymethyl)phenyl]pent-1-enyl]-5-oxocyclopentyl]sulfanylpropylsulfanyl]acetic acid (ONO-AE1-329); methyl4-[2-[(1R,2R,3R)-3-hydroxy-2-[(E,3S)-3-hydroxy-4-[3-(methoxymethyl)phenyl]but-1-enyl]-5-oxocyclopentyl]ethylsulfanyl]butanoate (ONO-4819); 16-(3-Methoxymethyl)phenyl-\acute{ω}-tetranor-5-thiaPGE1; 5-{3-[(2S)-2-{(3R)-3-hydroxy-4-[3-(trifluoromethyl)phenyl]butyl}-5-oxopyrrolidin-1-yl]propyl]thiophene-2-carboxylate (PF-04475270); APS-999 Na; [4′-[3-butyl-5-oxo-1-(2-trifluoromethyl-phenyl)-1,5-dihydro-[1,2,4]triazol-4-ylmethyl]-biphenyl-2-sulfonic acid (3-methyl-thiophene-2-carbonyl)-amide]; ((Z)-7-{(1R,4S,5R)-5-[(E)-5-(3-chloro-benzo[b]thiophene-2-yl)-3-hydroxy-pent-1-enyl]-4-hydroxy-3,3-dimethyl-2-oxo-cyclopentyl})-hept-5-enoic acid); Linoleic Acid; 13(s)-HODE; LY171883; Mead Acid; Eicosatrienoic Acid; Epoxyeicosatrienoic Acid; ONO-259; Cay1039; Butaprost; Sulprostone; CAY10399; ONO_8815Ly; ONO-AE1-259; and CP-533,536.

In some embodiments, the glucocorticoid is selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol.

In some embodiments, the prostaglandin pathway agonist is dmPGE2 and the glucocorticoid is dexamethasone.

In some embodiments, the isolated population or subpopulation of T lymphocytes has been contacted with the agent for at least about one hour, at least about one hour to about 24 hours, at least about one hour to about 12 hours, at least about one hour to about six hours, at least about one hour to about four hours, at least about two hours to about four hours. In some aspects, the isolated population or subpopulation of T lymphocytes has been contacted with the agent for about two hours or about four hours.

In some embodiments, the isolated population or subpopulation of T lymphocytes has been contacted with the agent at between about 24° C. to about 39° C. In some aspects, the isolated population or subpopulation of T lymphocytes has been contacted with the agent at about 37° C.

In some embodiments, the expression of a signature gene in the treated isolated population or subpopulation of T lymphocytes is increased by at least about two fold compared to a noncontacted population or subpopulation of T lymphocytes. In some aspects, the signature gene is selected from the group consisting of BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, IL21R, MCAM, SOCS1, TNFSF4, TOB1, TSC22D3, FGL2, NR4A2, AREG, TGFB1, CD55, THFAIP3, and CXCR4. In some aspects, the signature gene is selected from the group consisting of ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2. In some embodiments, the signature gene is selected from the group consisting of FGL2, NR4A2, AREG, TGFB1, CD55, CCR4, THFAIP3, NLRP3, CTLA4, and CXCR4. In some other embodiments, the signature gene is selected from the group consisting of Plaur, Fosl2, Ccr8, Areg, Nr4a2, Pdcd1, Nr4a3, and Ctla4.

In some embodiments, the signature genes include at least two, three, four, five, ten, fifteen, or all of the signature genes as disclosed herein. In some embodiments, the expression of the signature gene is increased by at least 3, 5, or 10 fold compared to a noncontacted population or subpopulation of T lymphocytes.

In some embodiments, the isolated population or subpopulation of T lymphocytes has an increased level of PD-1 at a cell surface compared to a noncontacted population or subpopulation of T lymphocytes. In some embodiments, the isolated population or subpopulation of T lymphocytes has a decreased level of ICOS or 41BB at a cell surface compared to a noncontacted population or subpopulation of T lymphocytes. In some embodiments, the treated isolated population or subpopulation of T lymphocytes has altered proliferation. In some embodiments, the treated isolated population or subpopulation of T lymphocytes having altered proliferation comprises CD4+ and/or CD8+ cells. The altered proliferation can be decreased proliferation compared to a noncontacted population or subpopulation of T lymphocytes. In some embodiments, the treated isolated population or subpopulation of T lymphocytes has a decreased allogeneic response compared to a noncontacted population or subpopulation of T lymphocytes. In some embodiments, the treated isolated population or subpopulation of T lymphocytes has reduced ability for allo-activation compared to a noncontacted population or subpopulation of T lymphocytes. In some embodiments, the treated isolated population or subpopulation of T lymphocytes has reduced ability for activation in response to an allo-antigen. In some embodiments, the treated isolated population or subpopulation of T lymphocytes has increased persistence compared to a noncontacted population or subpopulation of T lymphocytes. In some embodiments, the treated population or subpopulation of T lymphocytes has decreased production of cytokine including, but not limited to, interferon gamma and/or interleukin 17, compared to an untreated population or subpopulation of T lymphocytes. In some embodiments, the treated population or subpopulation of T lymphocytes has increased production of less inflammatory cytokine or anti-inflammatory cytokine compared to an untreated population or subpopulation of T lymphocytes. In some embodiments, the treated isolated population or subpopulation of T lymphocytes has increased production of interleukin 4 and/or interleukin 10 compared to an untreated population or subpopulation of T lymphocytes.

In some embodiments, the treated isolated population or subpopulation of T lymphocytes comprises increased percentage or number of Treg cells. In some embodiments, the treated isolated population or subpopulation of T lymphocytes comprises increased percentage or number of CD4+CD25^{hi} CD127^{lo}Foxp3⁺ cells. In some embodiments, the treated isolated population or subpopulation of T lymphocytes comprises cells having an increased level of one or more Treg effector molecules. In some embodiments, the one or more enhanced Treg effector molecules is selected from the group consisting of FGL2, NR4A2, AREG, TGFB1, CD55, CCR4, THFAIP3, NLRP3, CTLA4, and CXCR4. In some other embodiments, the one or more enhanced Treg effector molecules is selected from the group consisting of Plaur, Fosl2, Ccr8, Areg, Nr4a2, Pdcd1, Nr4a3, and Ctla4.

In some embodiments, the treated isolated population or subpopulation of T lymphocytes comprises Treg cells having improved survival. In some embodiments, the treated isolated population or subpopulation of T lymphocytes comprises Treg cells expressing an increased level of genes for attenuation of GvHD. In some embodiments, the treated isolated population or subpopulation of T lymphocytes comprises Treg cells expressing an increased level of genes for protecting against tissue injuries. In some embodiments, the treated isolated population or subpopulation of T lymphocytes comprises Treg cells expressing an increased level of genes for suppressor function.

In some embodiments, the present invention provides a pharmaceutical composition having the treated isolated population or subpopulation of T lymphocyte. The pharmaceutical composition can be washed with a buffer substantially free of the modulating agent.

In some embodiments, the present invention also provides a method of administering the isolated population or subpopulation of T lymphocytes, or the pharmaceutical composition to a subject in need of cell therapy. The subject can be a candidate for bone marrow or stem cell transplantation. In some embodiments, the subject has received bone marrow ablative or non-myeolablative chemotherapy or radiation therapy.

In some embodiments, the subject has a hyperproliferative disorder or a cancer. In some aspects, the subject has a hyperproliferative disorder or cancer of hematopoietic system, such as leukemia, lymphoma, or myeloma.

In some embodiments, the subject has a solid tumor, which can be breast cancer, ovarian cancer, brain cancer, prostate cancer, lung cancer, colon cancer, skin cancer, liver cancer, pancreatic cancer, or sarcoma.

In some embodiments, the subject has a virus infection or a disease associated with virus infection. In some aspects, the disease associated with virus infection is HIV/AIDS, Cervical cancer, Hepatitis, Hodgkin's lymphoma, or Influenza.

### BRIEF DESCRIPTION OF THE DRAWINGS

FIG. 1 shows the elevated expression levels of key genes in CD3+ T cells modulated by dmPGE2.

FIG. 2 shows the elevated expression levels of key genes in CD3+ T cells modulated by dmPGE2 and Dexamethasone. Results shown are the mean values of CD3+ T cells from three individual donors.

FIG. 3 shows the Mixed Lymphocyte Reaction Assay (MLR) to monitor allogeneic responsiveness. A. CD4+ and B. CD8+ T cells were enumerated at the end of a 5 day co-culture with mismatched peripheral blood mononuclear cells (“PBMC”).

FIG. 4 shows the cytokine release from T cells after stimulation for 5 days. A. Interferon gamma, B. Interleukin 4, and C. Interleukin 10 were monitored by intracellular cytokine staining using flow cytometry.

FIG. 5 shows the surface expression of key proteins on T cells after stimulation for 5 days. A. PD-1, B. ICOS and C. 41BB were monitored by flow cytometry and reported as mean fluorescence intensity.

FIG. 6 shows T regulatory cell number after modulation and stimulation (A), and in differentiation media (B) for 5 days. T regulator cells were identified by flow cytometry as CD4+/CD25hi/CD127^{lo}/Foxp3+.

FIG. 7 shows differential gene expression in Treg cells treated with dmPGE2 and dexamethasone compared to untreated Treg cells. A. human Treg cells; B. mouse Treg cells.

### DETAILED DESCRIPTION OF THE INVENTION

The present invention provides modulated T lymphocytes and methods and compositions for making and using the same. The modulated T lymphocytes can have improved properties for cell based therapies. In one embodiment, T lymphocytes are treated ex vivo with an agent that is a prostaglandin pathway agonist, a glucocorticoid, or a combination thereof.

As used herein, the articles “a,” “an,” and “the” refer to one or to more than one of the grammatical object of the article. By way of example, a T lymphocyte means one T lymphocyte or more than one T lymphocytes.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T lymphocyte can be any T lymphocyte, such as a cultured T lymphocyte, e.g., a primary T lymphocyte, or a T lymphocyte from a cultured T cell line, e.g., Jurkat, SupT1, etc., or a T lymphocyte obtained from a mammal. The T lymphocyte can be CD3+ cells. The T lymphocyte can be any type of T lymphocyte and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. A T lymphocyte can be T regulatory cell, which includes nTregs (natural Tregs), iTregs (inducible Tregs), CD8⁺ Treg, Tr1 regulatory cells, and Th3 cells. Additional types of helper T cells include cells such as Th3 (Treg), Th17, Th9, or Tfh cells. Additional types of memory T cells include cells such as central memory T cells (T_{CM} cells), effector memory T cells (T_{EM} cells and T_{EMRA} cells). The T lymphocyte can also refer to a genetically engineered T lymphocyte, such as a T lymphocyte modified to express a T cell receptor (TCR) or a chimeric antigen receptor (CAR). The T lymphocyte can also be differentiated from a stem cell, definitive hemogenic endothelium, a CD34+ cell, a HSC (hematopoietic stem and progenitor cell), a hematopoietic multipotent progenitor cell, or a T cell progenitor cell.

As used herein, the term “isolated” or the like when used in reference to a cell is intended to mean a cell that is substantially free of at least one component as the referenced cell is found in nature. The term includes a cell that is removed from some or all components as it is found in its natural environment. The term also includes a cell that is removed from at least one, some or all components as the cell is found in non-naturally occurring environments. Therefore, an isolated cell is partly or completely separated from other substances as it is found in nature or as it is grown, stored or subsisted in non-naturally occurring environments. Specific examples of isolated cells include partially pure cells, substantially pure cells and cells cultured in a medium that is non-naturally occurring. As used herein, the term “purify” or the like refers to increase purity. For example, the purity can be increased to at least 50%, 60%, 70%, 80%, 90%, 95%, 99%, or 100%.

As used herein, the term “population” when used with reference to T lymphocytes refers to a group of cells including two or more T lymphocytes. The isolated population of T lymphocytes can have only one type of T lymphocyte, or two or more types of T lymphocyte. The isolated population of T lymphocytes can be a homogeneous population of one type of T lymphocyte or a heterogeneous population of two or more types of T lymphocyte. The isolated population of T lymphocytes can also be a heterogeneous population having T lymphocytes and at least a cell other than a T lymphocyte, e.g., a B cell, a macrophage, a neutrophil, an erythrocyte, a hepatocyte, an endothelial cell, an epithelial cells, a muscle cell, a brain cell, etc. The heterogeneous population can have from 0.01% to about 100% T lymphocyte. Accordingly, an isolated population of T lymphocytes can have at least 50%, 60%, 70%, 80%, 90%, 95%, 98%, or 99% T lymphocytes. The isolated population of T lymphocytes can include only one type of T lymphocytes, or a mixture of more than one type of T lymphocytes. The isolated population of T lymphocytes can include one or more, or all of, the different types of T lymphocytes, including but not limited to those disclosed herein. An isolated population of T lymphocytes can include all known types of T lymphocytes. In an isolated population of T lymphocytes that includes more than one type of T lymphocytes, the ratio of each type of T lymphocyte can range from 0.01% to 99.99%. The isolated population also can be a clonal population of T lymphocytes, in which all the T lymphocytes of the population are clones of a single T lymphocyte.

The isolated population of T lymphocytes obtained from a natural source, such as human peripheral blood or cord blood, refers to a population of cells that has been enriched for T lymphocytes from the natural source. Accordingly, the proportion of T lymphocytes in an isolated population of T lymphocytes is higher as compared to the proportion of T lymphocytes in the natural source prior to being enriched. The T lymphocytes can be enriched by a sorting or selection process as described herein or by other methods known in the art. The proportion of T lymphocytes in the isolated population can be higher than the proportion of T lymphocytes in the natural source by at least about 10%, about 20%, about 30%, about 40%, about 50%, about 60%, about 70%, about 80%, about 85%, about 90%, or about 95%. The isolated population of T lymphocytes can be a population of cells enriched for T lymphocytes in general, or one or more specific types of T lymphocytes.

As used herein, the term “subpopulation” when used in reference to T lymphocytes refers to a population of T lymphocytes that includes less than all types of T lymphocytes that are found in nature.

As used herein, the term “differentiate,” “differentiation,” or the like refers to the process by which an unspecialized (or uncommitted) or less specialized cell acquires the features of a specialized cell such as, for example, a blood cell or a muscle cell. A differentiated or differentiation-induced cell is one that has taken on a more specialized (or committed) position within the lineage of a cell. A cell is committed when it has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or, “iPSCs,” refers to stem cells produced from differentiated adult cells that have been induced or changed (i.e. reprogrammed) into cells capable of differentiating into tissues of all three germ or dermal layers: mesoderm, endoderm, and ectoderm.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta and are not totipotent.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “exogenous” is intended to mean that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “ex vivo” refers to activities that take place outside an organism, such as experimentation or measurements done in or on living tissue in an artificial environment outside the organism, preferably with minimum alteration of the natural conditions. The “ex vivo” procedures can involve living cells or tissues taken from an organism and cultured in a laboratory apparatus, usually under sterile conditions, and typically for a few hours or up to about 24 hours, but including up to 48 or 72 hours, depending on the circumstances. Such tissues or cells can also be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo. Meanwhile, an “in vivo” activity takes place inside an organism, such as cell engraftment, cell homing, self-renewal of cells, and expansion of cells.

As used herein, the term “in vitro” refers to activities performed or taking place in a test tube, culture dish, or elsewhere outside a living organism.

As used herein, the terms “agent,” “modulating agent,” and “modulator” are used interchangeably herein to refer to a compound or molecule capable of modifying gene expression profile or a biological property of a T lymphocyte. The agent can be a single compound or molecule, or a combination of more than one compound or molecule. Exemplary agents include, for example, compounds capable of stimulating the prostaglandin pathway, e.g., prostaglandin pathway agonists, glucocorticoids or combinations thereof.

As used herein, the terms “contact,” “treat,” or “modulate,” when used in reference to a T lymphocyte, are used interchangeably herein to refer to culturing, incubating or exposing a T lymphocyte ex vivo with one or more of the agents disclosed herein.

As used herein, a “noncontacted” or an “untreated” cell is a cell that has not been treated, e.g., cultured, contacted, or incubated with an agent other than a control agent. Cells contacted with a control agent, such as DMSO, or contacted with another vehicle are examples of noncontacted cells.

As used herein, the term “prostaglandin pathway agonist” refers to an agent that stimulates prostaglandin cell signaling pathways. The prostaglandin pathway agonist can be a PGE receptor agonist, such as an agent that binds and activates one or more of the PGE₂ EP₁, PGE₂ EP₂, PGE₂ EP₃, or PGE₂ EP₄ receptor.

As used herein, the terms “prostaglandin E₂” or “PGE₂” include, without limitation, any naturally occurring or chemically synthesized PGE₂ molecule, as well as “analogues” thereof.

As used herein, the term “analogue” refers to a chemical molecule that is similar to another chemical substance, e.g., PGE₂, in structure and function, differing structurally by one single element or group, or more than one group (e.g., 2, 3, or 4 groups) if it retains the same function as the parental chemical. Such modifications are routine to persons skilled in the art, and include, for example, additional or substituted chemical moieties, such as esters or amides of an acid, protecting groups such as a benzyl group for an alcohol or thiol, and tert-butoxylcarbonyl groups for an amine. Also included are modifications to alkyl side chains, such as alkyl substitutions (e.g., methyl, dimethyl, ethyl, etc.), modifications to the level of saturation or unsaturation of side chains, and the addition of modified groups such as substituted phenyl and phenoxy. Analogues can also include conjugates, such as biotin or avidin moieties, enzymes such as horseradish peroxidase and the like, and including radio-labeled, bioluminescent, chemoluminescent, or fluorescent moieties. Also, moieties can be added to the agents described herein to alter their pharmacokinetic properties, such as to increase half-life in vivo or ex vivo, or to increase their cell penetration properties, among other desirable properties. Also included are prodrugs, which are known to enhance numerous desirable qualities of pharmaceuticals (e.g., solubility, bioavailability, manufacturing, etc.) (see, e.g., WO/2006/047476 for exemplary EP agonist prodrugs, which is hereby incorporated by reference for its disclosure of such agonists).

As used herein, the term “signature gene” refers to any gene that can distinguish a group of cells with particular biological properties or therapeutic potentials from existing cells in the art and/or control, vehicle, or untreated T lymphocytes by its expression level. For clarity, signature genes do not include housekeeping genes.

As used herein, “gene expression” refers to the relative levels of expression and/or pattern of expression of a gene in a biological sample, such as an isolated T lymphocyte, or population of cells having T lymphocytes.

As used herein, the term “increase” or “enhance” refers to the ability of an agent to produce or cause a greater physiological response (i.e., downstream effects) in a cell, as compared to the response caused by either vehicle or a control molecule/composition, e.g., increased production of interleukin 4 or interleukin 10 by an isolated population of T lymphocytes. The increase can be an increase in gene expression as a result of increased signaling through certain cell signaling pathways. An “increased” amount is typically a statistically significant amount, and can include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) compared to the response produced by vehicle (the absence of an agent) or a control composition.

As used herein, the term “decrease” refers to the ability of an agent to produce or cause a lesser physiological response (i.e., downstream effects) in a cell, as compared to the response caused by either vehicle or a control molecule/composition. The decrease can be a decrease in gene expression, a decrease in cell signaling, or a decrease in cell proliferation. An “decreased” amount is typically a “statistically significant” amount, and can include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle (the absence of an agent) or a control composition.

As used herein, the terms “substantially free of,” when used to describe a composition, such as a cell population or culture media, refers to a composition that is free of a specified substance, such as, 95% free, 96% free, 97% free, 98% free, 99% free of the specified substance, or is undetectable as measured by conventional means. Similar meaning can be applied to the term “absence of,” where referring to the absence of a particular substance or component of a composition.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. The range of quantity, level, value, number, frequency, percentage, dimension, size, amount, weight or length can be ±15%, ±10%, ±9%, ±8%, ±7%, ±6%, ±5%, ±4%, ±3%, ±2%, or 1% about a reference quantity, level, value, number, frequency, percentage, dimension, size, amount, weight or length.

As used herein, the term “subject,” refers to a mammal. A subject can be a human or a non-human mammal such as a dog, cat, bovid, equine, mouse, rat, rabbit, or transgenic species thereof.

As used herein, the term “treat,” and the like, when used in reference to a subject, refer to obtaining a desired pharmacologic and/or physiologic effect, including without limitation achieving an improvement or elimination of the symptoms of a disease. The effect can be prophylactic in terms of completely or partially preventing a disease or symptom thereof and/or can be therapeutic in terms of achieving an improvement or elimination of symptoms, or providing a partial or complete cure for a disease and/or adverse affect attributable to the disease. The term treatment includes any treatment of a disease in a mammal, particularly in a human, and includes: (a) preventing the disease from occurring in a subject which can be predisposed to the disease but has not yet been diagnosed as having it; (b) inhibiting the disease, or arresting its development; (c) relieving the disease, or causing regression of the disease, or to completely or partially eliminate symptoms of the disease; and (d) restoring the individual to a pre-disease state, such as reconstituting the hematopoietic system.

The present invention provides an isolated population or subpopulation of T lymphocytes, as well as a composition having an isolated population or subpopulation of T lymphocytes, that has been contacted ex vivo with one or more agents to improve its therapeutic potential when used in a cell based therapy. The ex vivo treatment can modify the biological properties of the T lymphocytes to improve their therapeutic potential or reduce the relapse rate of the disease. For example, the ex vivo treatment can decrease the risk for the T lymphocyte to produce an allogeneic response and increase their persistence in the body of a subject. The ex vivo treatment can also alter the ratios of different types of T lymphocytes in the immune reconstitution, and produce better long term effects. For example, the treatment can increase the ratio of naive T lymphocytes, central memory T cells, or both in the immune reconstitution. The ex vivo treatment can increase percentage or number of Treg cells in the isolated population or subpopulation of T lymphocytes. The ex vivo treatment can increase the level of one or more Treg effector molecules in the cells comprised in the isolated population or subpopulation of T lymphocytes. In some embodiments, the one or more enhanced Treg effector molecules is selected from the group consisting of FGL2, NR4A2, AREG, TGFB1, CD55, CCR4, THFAIP3, NLRP3, CTLA4, and CXCR4. In some other embodiments, the one or more enhanced Treg effector molecules is selected from the group consisting of Plaur, Fosl2, Ccr8, Areg, Nr4a2, Pdcd1, Nr4a3, and Ctla4. The ex vivo treatment can improve survival of Treg cells comprised in the isolated population or subpopulation of T lymphocytes. The ex vivo treatment can increase the expression level of genes relating to GvHD attenuation in Treg cells comprised in the isolated population or subpopulation of T lymphocytes. The ex vivo treatment can increase the expression level of genes relating to tissue injury prevention in Treg cells comprised in the isolated population or subpopulation of T lymphocytes. The ex vivo treatment can increased the expression level of genes relating to suppressive functions in Treg cells comprised in the isolated population or subpopulation of T lymphocytes.

The isolated population or subpopulation of T lymphocytes can have any T lymphocytes, such as cultured T lymphocytes, primary T lymphocytes, T lymphocytes that are in vitro differentiated from a stem cell or a progenitor cell, or recombinant T lymphocytes that have an exogenous polynucleotide.

In some embodiments, the present invention provides an isolated population or subpopulation of T lymphocytes that have been contacted ex vivo with an agent, wherein the agent is a prostaglandin pathway agonist, a glucocorticoid, or a combination thereof.

In some embodiments, the T lymphocytes are human T lymphocytes. In some embodiments, the T lymphocytes are isolated from a human. The T lymphocytes can be any type of T lymphocyte and can be of any developmental stage, including but not limited to, CD4+/CD8+ double positive T cells, CD4+ T cells, CD8+ T cells, naive T cells, effector T cells, cytotoxic T cells, helper T cells, memory T cells, regulator T cells, Th0 cells, Th1 cells, Th2 cells, Th3 (Treg) cells, Th9 cells, Thαβ helper cells, Tfh cells, stem memory T_{SCM} cells, central memory T_{CM} cells, effector memory T_{EM} cells, effector memory T_{EMRA} cells, and gamma delta T cells, peripheral blood mononuclear cells (“PBMC”), peripheral blood leukocytes (“PBL”), tumor infiltrating lymphocytes (“TIL”), memory T cells, naive T lymphocytes, and the like.

In some embodiments, the isolated population or subpopulation of T lymphocytes include peripheral blood mononuclear cells (PBMC), peripheral blood leukocytes (PBL), or tumor infiltrating lymphocytes (TIL). In some embodiments, the isolated population or subpopulation of T lymphocytes are peripheral blood mononuclear cells (PBMC), peripheral blood leukocytes (PBL), or tumor infiltrating lymphocytes (TIL).

In some embodiments, the isolated population or subpopulation of T lymphocytes are selected from the group consisting of CD4+/CD8+ double positive T cells, CD4+ T cells, CD8+ T cells, naive T cells, effector T cells, cytotoxic T cells, helper T cells, memory T cells, regulator T cells, Th0 cells, Th1 cells, Th2 cells, Th3 (Treg) cells, Th9 cells, Thαβ helper cells, Tfh cells, stem memory T_{SCM} cells, central memory T_{CM} cells, effector memory T_{EM} cells, effector memory T_{EMRA} cells, and gamma delta T cells, or any combination thereof.

In some embodiments, the isolated population or subpopulation of T lymphocytes have CD4+/CD8+ double positive T cells. In some embodiments, the isolated population or subpopulation of T lymphocytes have CD4+ T cells. In some embodiments, the isolated population or subpopulation of T lymphocytes have CD8+ T cells. In some embodiments, the isolated population or subpopulation of T lymphocytes have naive T cells. In some embodiments, the isolated population or subpopulation of T lymphocytes have effector T cells. In some embodiments, the isolated population or subpopulation of T lymphocytes have cytotoxic T cells. In some embodiments, the isolated population or subpopulation of T lymphocytes have helper T cells. In some embodiments, the isolated population or subpopulation of T lymphocytes have memory T cells. In some embodiments, the isolated population or subpopulation of T lymphocytes have regulator T cells. In some embodiments, the isolated population or subpopulation of T lymphocytes have Th0 cells. In some embodiments, the isolated population or subpopulation of T lymphocytes have Th1 cells. In some embodiments, the isolated population or subpopulation of T lymphocytes have Th2 cells. In some embodiments, the isolated population or subpopulation of T lymphocytes have Th3 (Treg) cells. In some embodiments, the isolated population or subpopulation of T lymphocytes have Th9 cells. In some embodiments, the isolated population or subpopulation of T lymphocytes have Thαβ helper cells. In some embodiments, the isolated population or subpopulation of T lymphocytes have Tfh cells. In some embodiments, the isolated population or subpopulation of T lymphocytes have stem memory T_{SCM} cells. In some embodiments, the isolated population or subpopulation of T lymphocytes have central memory T_{CM} cells. In some embodiments, the isolated population or subpopulation of T lymphocytes have effector memory T_{EM} cells. In some embodiments, the isolated population or subpopulation of T lymphocytes have effector memory T_{EMRA} cells. In some embodiments, the isolated population or subpopulation of T lymphocytes have gamma delta T cells. In some other embodiments, the isolated population or subpopulation of T lymphocytes have any combination of the different types of T lymphocytes as disclosed herein or known in the art.

As a person of ordinary skill in the art would understand, different types of T lymphocyte or combinations of different types of T lymphocyte can have different biological properties and function. Depending on specific conditions of the patients or subjects in need of a cell therapy, or a T cell therapy, different types of T lymphocyte or combinations of different types of T lymphocyte can be used. A person of ordinary skill in the art can select the proper type of T lymphocyte, or combination of different types of T lymphocyte, as needed.

In some embodiments, the isolated population or subpopulation of T lymphocytes have at least 10%, 20%, 30%, 40%, 50%, 60%, 70%, 80%, 90%, 95%, 98%, or 99% of T lymphocytes. In some embodiments, the isolated population or subpopulation of T lymphocytes have 10%, 20%, 30%, 40%, 50%, 60%, 70%, 80%, 90%, 95%, 98%, or 99% of T lymphocytes. In some embodiments, the isolated population or subpopulation of T lymphocytes have only one specific type of the T lymphocytes disclosed herein.

In some embodiments, the present invention provides an isolated subpopulation of T lymphocytes that have been contacted ex vivo with an agent, wherein the agent is a prostaglandin pathway agonist, a glucocorticoid, or a combination thereof, and wherein the isolated subpopulation of T lymphocytes having two or more types of T lymphocytes selected from the group consisting of CD4+/CD8+ double positive T cells, CD4+ T cells, CD8+ T cells, naive T cells, effector T cells, cytotoxic T cells, helper T cells, memory T cells, regulator T cells, Th0 cells, Th1 cells, Th2 cells, Th3 (Treg) cells, Th9 cells, Thαβ helper cells, Tfh cells, stem memory T_{SCM} cells, central memory T_{CM} cells, effector memory T_{EM} cells, effector memory T_{EMRA} cells, and gamma delta T cells.

In some embodiments, the isolated population or subpopulation of T lymphocytes have a mixture of at least 2, 3, 4, 5, 6, 7, 8, 9, 10, 11, 12, 13, 14, 15, 16, 17, 18, 19, 20, or 21 different types of T lymphocytes as disclosed herein or known in the art. In some aspects, the isolated population or subpopulation of T lymphocytes have a mixture of 2, 3, 4, 5, 6, 7, 8, 9, 10, 11, 12, 13, 14, 15, 16, 17, 18, 19, 20, or 21 types different types of T lymphocytes as disclosed herein or known in the art. In some aspects, the isolated population or subpopulation of T lymphocytes include more than one types of T lymphocytes, and the ratio of each type of T lymphocytes can range from 0.01% to 99.99%. For example, in an isolated population or subpopulation of T lymphocytes that includes two types of T lymphocytes, the ratio of the two types of T lymphocytes can be any ratio between 0.01: 99.99 to 99.99: 0.01, including such as, 1: 99; 5: 95; 10: 90; 20: 80; 30: 70; 40: 60; 50:50; 60: 40; 70: 30; 80: 20; 90: 10; 95: 5; 99:1. As such, a person of ordinary skill in the art would understand that in the isolated population or subpopulation of different types of T lymphocytes of the invention, different types of T lymphoctyes can make up any percentage of the total T lymphocytes, ranging from 0.01%-99.99%, and have different ratios with respect to each other.

In some embodiments, the isolated population or subpopulation of T lymphocytes are CD4+/CD8+ double positive T cells. In some embodiments, the isolated population or subpopulation of T lymphocytes are CD4+ T cells. In some embodiments, the isolated population or subpopulation of T lymphocytes are CD8+ T cells. In some embodiments, the isolated population or subpopulation of T lymphocytes are naive T cells. In some embodiments, the isolated population or subpopulation of T lymphocytes are effector T cells. In some embodiments, the isolated population or subpopulation of T lymphocytes are cytotoxic T cells. In some embodiments, the isolated population or subpopulation of T lymphocytes are helper T cells. In some embodiments, the isolated population or subpopulation of T lymphocytes are memory T cells. In some embodiments, the isolated population or subpopulation of T lymphocytes are regulator T cells. In some embodiments, the isolated population or subpopulation of T lymphocytes are Th0 cells. In some embodiments, the isolated population or subpopulation of T lymphocytes are Th1 cells. In some embodiments, the isolated population or subpopulation of T lymphocytes are Th2 cells. In some embodiments, the isolated population or subpopulation of T lymphocytes are Th3 (Treg) cells. In some embodiments, the isolated population or subpopulation of T lymphocytes are Th9 cells. In some embodiments, the isolated population or subpopulation of T lymphocytes are Thαβ helper cells. In some embodiments, the isolated population or subpopulation of T lymphocytes are Tfh cells. In some embodiments, the isolated population or subpopulation of T lymphocytes are stem memory T_{SCM} cells. In some embodiments, the isolated population or subpopulation of T lymphocytes are central memory T_{CM} cells. In some embodiments, the isolated population or subpopulation of T lymphocytes are effector memory T_{EM} cells. In some embodiments, the isolated population or subpopulation of T lymphocytes are effector memory T_{EMRA} cells. In some embodiments, the isolated population or subpopulation of T lymphocytes are gamma delta T cells.

In some embodiments, the isolated population or subpopulation of T lymphocytes is differentiated in vitro from a stem cell, a definitive hemogenic endothelium, a CD34+ cell, a HSC (hematopoietic stem and progenitor cell), a hematopoietic multipotent progenitor cell, or a T cell progenitor cell.

In some embodiments, the isolated population or subpopulation of T lymphocytes have an exogenous nucleic acid. In some aspects, the exogenous nucleic acid can encode a TCR or a CAR.

In some embodiments, the present invention provides an isolated population of T lymphocytes that has been contacted ex vivo with an agent comprising a prostaglandin pathway agonist and a glucocorticoid.

The isolated population or subpopulation of T lymphocytes can be obtained from a human or mammal other than a human. Examples of such non-human mammals include, but are not limited to rabbit, horse, bovine, sheep, pigs, dogs, cats, mice, rats, and transgenic species thereof. The isolated population or subpopulation of T lymphocytes can be obtained from a number of sources, including but not limited to peripheral blood, bone marrow, lymph node tissue, cord blood, thymus tissue, tissue from a site of infection, ascites, pleural effusion, spleen tissue, and tumors. The bone marrow can be obtained from femurs, iliac crest, hip, ribs, sternum, and other bones. In addition, the T lymphocyte lines available in the art can also be used, such as Jurkat, SupT1, and others.

In certain embodiments of the present invention, the isolated population or subpopulation of T lymphocytes can be separated from a unit of blood using any number of techniques known to the skilled artisan, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis can be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and can lack magnesium or can lack many if not all divalent cations. As those of ordinary skill in the art would readily appreciate a washing step can be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells can be resuspended in a variety of biocompatible buffers, such as, for example, Ca-free, Mg-free PBS, PlasmaLyte A, or other saline solution with or without buffer. Alternatively, the undesirable components of the apheresis sample can be removed and the cells directly resuspended in culture media.

In another embodiment, the isolated population or subpopulation of T lymphocytes are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD28+, CD4+, CD8+, CD45RA+, or CD45RO+ T cells, can be further isolated by positive or negative selection techniques. For example, in one embodiment, the isolated population or subpopulation of T lymphocytes are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T lymphocytes from patients with leukemia, use of longer incubation times, such as 24 hours, can increase cell yield. Longer incubation times can be used to isolate T lymphocytes in any situation where there are few T lymphocytes as compared to other cell types, such in isolating tumor infiltrating lymphocytes (TIL) from tumor tissue or from immunocompromised individuals. Further, use of longer incubation times can increase the efficiency of capture of CD8+ T lymphocytes. Thus, by simply shortening or lengthening the time T lymphocytes are allowed to bind to the CD3/CD28 beads and/or by increasing or decreasing the ratio of beads to T lymphocytes (as described further herein), specific populations or subpopulations of T lymphocytes can be further selected for or against at culture initiation or at other time points during the process. Additionally, by increasing or decreasing the ratio of anti-CD3 and/or anti-CD28 antibodies on the beads or other surface, specific populations or subpopulations of T lymphocytes can be preferentially selected for or against at culture initiation or at other desired time points. The skilled artisan would recognize that multiple rounds of selection can also be used in the context of this invention. In certain embodiments, it can be desirable to perform the selection procedure and use the “unselected” cells in the activation and expansion process. “Unselected” cells can also be subjected to further rounds of selection.

Enrichment of a population or subpopulation of T lymphocytes by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD3+ cells by negative selection, a monoclonal antibody cocktail typically includes antibodies to CD14, CD20, CD1 b, CD16, HLA-DR, CD4 and CD8. In certain embodiments, it can be desirable to enrich for or positively select for regulatory T lymphocytes which typically express CD4+, CD25+, CD62L^{hi}, GITR+, and FoxP3+. Alternatively, in certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, the volume can be significantly decreased in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that weakly express target antigens of interest, such as CD28− T lymphocytes, or from samples where there are many tumor cells present (i.e., leukemic blood, tumor tissue, etc.). Such populations of cells can have therapeutic value and would be desirable to obtain. For example, using high concentration of cells allows more efficient selection of CD8+ T cells that normally have weaker CD28 expression.

It can be desirable to use lower concentrations of cells. By significantly diluting the mixture of T lymphocytes and surface (e.g., particles such as beads), interactions between the particles and cells is minimized. This selects for cells that express high amounts of desired antigens to be bound to the particles. For example, CD4+ T lymphocytes express higher levels of CD28 and are more efficiently captured than CD8+ T lymphocytes in dilute concentrations. In one embodiment, the concentration of cells used is 5×10⁶ cells/ml. In other embodiments, the concentration used can be from about 1×10⁵/ml to 1×10⁶ cells/ml, and any integer value in between.

In other embodiments, the cells can be incubated on a rotator for varying lengths of time at varying speeds at either 2-10° C., or at room temperature.

T lymphocytes for modulation can also be frozen after a washing step. The freeze and subsequent thaw step provides a more uniform product by removing granulocytes and to some extent monocytes in the cell population. After the washing step that removes plasma and platelets, the cells can be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing can be used as well as uncontrolled freezing immediately at −20° C. or in liquid nitrogen.

In certain embodiments, cryopreserved cells are thawed and washed as described herein and are immediately subject to modulation or allowed to rest for a period of time prior to modulation using the methods of the present invention. The period of time can be about 15 minutes, about 30 minutes, about 1 hour, or longer than 1 hour.

The blood samples or apheresis product from a subject can be collected at a time period prior to when the T lymphocytes as described herein are isolated. As such, the source of the cells to be modulated can be collected at any time point necessary, and desired cells, such as T lymphocytes, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T lymphocytes can be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, the isolated population or subpopulation of T lymphocytes can be differentiated from a stem cell or a progenitor cell. The stem cell can be a pluripotent stem cell, such as induced pluripotent stem cells (iPSCs) and embryonic stem cells (ESCs). The iPSC is a non-naturally occurring reprogrammed pluripotent cell. Once the cells of a subject have been reprogrammed to a pluripotent state, the cells can then be programmed to a desired cell type, such as a T lymphocyte. In some embodiments, the iPSC is differentiated to a T lymphocyte by a multi-stage differentiation platform wherein cells from various stages of development can be induced to assume a hematopoietic phenotype, ranging from mesodermal stem cells, definitive hemogenic endothelium, CD34 cells, hematopoietic stem and progenitor cells, hematopeoietic multipotent progenitor cells, T cell progenitor cells, to fully differentiated T lymphocytes.

Various strategies are being pursued to induce pluripotency, or increase potency, in cells (Takahashi, K., and Yamanaka, S., Cell 126, 663-676 (2006); Takahashi et al., Cell 131, 861-872 (2007); Yu et al., Science 318, 1917-1920 (2007); Zhou et al., Cell Stem Cell 4, 381-384 (2009); Kim et al., Cell Stem Cell 4, 472-476 (2009); Yamanaka et al., 2009; Saha, K., Jaenisch, R., Cell Stem Cell 5, 584-595 (2009)), and improve the efficiency of reprogramming (Shi et al., Cell Stem Cell 2, 525-528 (2008a); Shi et al., Cell Stem Cell 3, 568-574 (2008b); Huangfu et al., Nat Biotechnol 26, 795-797 (2008a); Huangfu et al., Nat Biotechnol 26, 1269-1275 (2008b); Silva et al., Plos Bio 6, e253. Doi: 10.1371/journal. Pbio. 0060253 (2008); Lyssiotis et al., PNAS 106, 8912-8917 (2009); Ichida et al., Cell Stem Cell 5, 491-503 (2009); Maherali, N., Hochedlinger, K., Curr Biol 19, 1718-1723 (2009b); Esteban et al., Cell Stem Cell 6, 71-79 (2010); and Feng et al., Cell Stem Cell 4, 301-312 (2009)), the disclosures of which are hereby incorporated by reference in their entireties.

In one embodiment, the multistage process can begin with a first stage wherein a pluripotent stem cell, such as an iPSC or ESC, is differentiated to a mesodermal stem cell. In one embodiment, the pluripotent cell is differentiated to a mesodermal stem cell by contacting the cell with at least one of a BMP pathway activator, a Wnt pathway activator, and an extracellular matrix protein. In a second stage, the mesodermal stem cell is differentiated to a hemogenic endothelium cell by maintaining the mesodermal stem cell's contact with at least one of the BMP pathway activator, the Wnt pathway activator, and the extracellular matrix protein, and further contacting the mesodermal stem cell with a TGFβ receptor inhibitor to produce a definitive hemogenic endothelial cell. In a third stage, the definitive hemogenic endothelial stem cell is differentiated to a hematopoietic stem cell by (i) maintaining the hemogenic endothelial cell's contact with at least one of the BMP pathway activator and the extracellular matrix protein, (ii) contacting the hemogenic endothelial cell with at least one hematopoietic specific cytokine, and/or (iii) removing at least one of the Wnt pathway activator and the TGFβ receptor inhibitor thereby producing a hematopoietic stem cell. Suitable hematopoietic specific cytokines for use with the invention include, but are not limited to, VEGF, SCF, FLT3L, IL15, IL3, IL6, IGF, TPO and combinations thereof. In a fourth stage, the hematopoietic stem cell can then differentiated to a more fully differentiated hematopoietic cell, including, but not limited to a T cell progenitor, or a B cell progenitor.

In one embodiment, the fourth stage includes differentiating the hematopoietic stem cell to a T cell progenitor by (i) maintaining the definitive hematopoietic stem cell's contact with the hematopoietic specific cytokine, except for VEGF and IL15, (ii) removing contact with at least one of the extracellular matrix protein and the BMP pathway activator, and/or (iii) contacting the hematopoietic stem cell with at least one of a Notch pathway activator and IL-2 and IL-7, thereby producing a T cell progenitor. In a fifth stage, the T cell progenitor is differentiated to a fully differentiated T cell by maintaining the T cell progenitor in culture conditions according to the preceding stage, with the exception of removing the BMP pathway activator.

In some embodiments, the differentiation of a stem cell can begin with the differentiation of a mesodermal stem cell, a definitive hemogenic endothelial cell, a hematopoietic stem cell, or a T cell progenitor.

In some embodiments, one or more of the stages of differentiation described above can be carried out under feeder free conditions. Such feeder free conditions include, but are not limited to, monolayer culture and suspension culture. In one aspect, the differentiation of a pluripotent cell to a mesodermal stem cell can be carried out under monolayer feeder free conditions without embryoid body (EB) formation. In another aspect, the differentiation of a mesodermal stem cell to a definitive hemogenic endothelial cell can be carried out under monolayer feeder free conditions. In another aspect, the differentiation of a definitive hemogenic endothelial cell to a hematopoietic stem cell can be carried out under monolayer feeder free conditions. In another aspect, the differentiation of a hematopoietic stem cell to a T cell progenitor is carried out under suspension feeder free conditions. In yet another aspect, the differentiation of a T cell progenitor to a fully differentiated T cell, is carried out under suspension feeder free conditions.

Accordingly, the present invention provides an isolated population or subpopulation of T lymphocytes obtained from any of the sources described above or by any of the methods described above, wherein the isolated population or subpopulation of T lymphocytes are contacted ex vivo with an agent that is a prostaglandin pathway agonist, a glucocorticoid, or a combination thereof under conditions sufficient to improve its therapeutic potential, such as to increase its persistence in the body when administered to a subject and/or to reduce its potential to initiate an allogeneic response. Without being limited to any particular theory or mechanism, contacting T lymphocytes with the agents described herein promotes anergy in the T lymphocytes and reduces their proliferation potential such that the T lymphocytes are able to persist in the body of a subject after being administered to the subject. This anergy and decrease in proliferation potential prevents exhaustion of the treated T lymphocytes and permits the cells to persist in the subject for a longer period of time and thereby provide a sustained therapeutic effect. Because the effects of the agents on the T lymphocytes can be temporary, anergy and decreased proliferation potential in the treated T lymphocytes is transitory and permits the T lymphocytes to become therapeutically active, against tumor or viral antigens for example, in a sustained, gradual manner.

The isolated population or subpopulation of T lymphocytes can also be genetically engineered for specific recognition of tumor antigens or viral antigens. The genetically engineered T lymphocytes can be used for treatment of cancers that include, but are not limited to, blood malignancies and solid tumors, or a disease associated with viral infection. In some embodiments, the isolated population or subpopulation of genetically engineered T lymphocytes are contacted ex vivo with an agent that is a prostaglandin pathway agonist, a glucocorticoid, or a combination thereof. The modulated population or subpopulation of T lymphocytes can have improved therapeutic potential.

In some embodiments, the T lymphocytes may be genetically engineered T lymphocytes. That is, the T lymphocytes may be modified to express an exogenous polynucleotide. The T lymphocytes can have an exogenous polynucleotide that improves the therapeutic efficacy of the T lymphocyte. The exogenous polynucleotide can encode a TCR. The exogenous polynucleotide can encode a CAR. In some embodiments, the T lymphocytes are genetically engineered to express a recombinant TCR of known specificity which recognizes cognate peptide-loaded major histocompatibility complexes (pMHC) of a tumor-associated antigen. In some embodiments, the T lymphocytes are genetically engineered to express the recombinant CAR, which in the extracellular part consists of an antibody with pre-defined binding specificity to a broad variety of targets and in the intracellular part of a T-cell activation domain.

In some embodiments, the T lymphocytes of the present invention are genetically engineered to express a recombinant TCR that is capable of direct recognition of tumor antigen and MHC expressing cancer cells. The TCR expressing T lymphocytes of the present invention are contacted ex vivo with agents under conditions sufficient to increase the engraftment and homing when transplanted to a recipient. In certain embodiments, the invention provides mixtures of cells expressing TCRs, or cells expressing more than one TCR described herein, that are specific for distinct cancer antigens, thus presenting cell populations that can be considered polyvalent with respect to the TCRs. As a person of ordinary skill in the art would understand, a recombinant TCR means a TCR that is expressed from a polynucleotide that was introduced into the cell, meaning prior to the introduction of the polynucleotide the TCR was not encoded by a chromosomal sequence in the cell. The methods and uses of T lymphocytes with recombinant TCRs are known in the art. See e.g., U.S. Pat. No. 8,008,438; WO2010088160; WO2012038055; US20100021468; WO2014160030; Robbins et al, J. Immuno., 180: 6116-6131(2008); Robbins et al, J. Clin. Onc., 29: 917-924 (2011), the disclosures of which are hereby incorporated by reference in their entireties.

The genetically modified T lymphocytes of the invention can be isolated cells, grown, expanded, or maintained in culture. In embodiments, the genetically modified T lymphocytes can have packaging plasmids, which, for example, provide some or all of the proteins used for transcription and packaging of an RNA copy of the expression construct into recombinant viral particles, such as pseudoviral particles. In embodiments, the expression vectors are transiently or stably introduced into cells. In embodiments, the expression vectors are integrated into the chromosome of cells used for their production. In embodiments, polynucleotides encoding the exogenous polynucleotides (e.g. TCRs and CARs) which are introduced into cells by way of an expression vector, such as a viral particle, are integrated into one or more chromosomes of the cells. Such cells can be used for propagation, or they can be cells that are used for therapeutic and/or prophylactic approaches. Such T lymphocytes include, but are not limited to, CD4+ T lymphocytes, CD8+ T lymphocytes, CD3+ T lymphocytes, and other types of T lymphocytes as described above, as well as their progenitor cells into which an exogenous gene expression construct has been introduced. The T lymphocytes can be from any source, including but not limited to autologous T lymphocytes and allogeneic T lymphocytes. The allogeneic T lymphocytes used for cell therapy can be either complete or partial HLA-match with the patient. It is also contemplated that the T lymphocytes may be mismatched for an intended subject due to the ability of the agents disclosed herein to produce anergy and avoid GvHD.

Expression vectors for use with embodiments of this disclosure can be any suitable expression vector. In embodiments, the expression vector includes a modified viral polynucleotide, such as from an adenovirus, a Sendai virus, a herpesvirus, or a retrovirus, such as a lentiviral vector. The expression vector is not restricted to recombinant viruses and includes non-viral vectors such as DNA plasmids and in vitro transcribed mRNA.

Genetically modified T lymphocytes may be modified by introducing an exogenous polynucleotide into a safe harbor locus so as to avoid disruption of the expression of endogenous genes in the cell thereby increasing stability of the cell. Safe harbor loci for genetically engineering T lymphocytes as disclosed herein include Rosa28 and AAVS1.

With respect to the polypeptides that are encoded by the polynucleotides described above, in certain aspects the invention provides functional TCRs which include a TCR α chain and a TCR β chain, wherein the two chains are present in a physical association with one another (e.g., in a complex) and are non-covalently joined to one another, or wherein the two chains are distinct polypeptides but are covalently joined to one another, such as by a disulfide or other covalent linkage that is not a peptide bond. Other suitable linkages can include, for example, substituted or unsubstituted polyalkylene glycol, and combinations of ethylene glycol and propylene glycol in the form of, for example, copolymers. In other embodiments, two polypeptides that constitute the TCR α and a TCR β chain can both be included in a single polypeptide, such as a fusion protein. In certain embodiments, the fusion protein includes a TCR α chain amino acid sequence and a TCR β chain amino acid sequence that have been translated from the same open reading frame (ORF), or distinct ORFs, or an ORF that contain a signal that results in non-continuous translation. In one embodiment, the ORF includes a P2A-mediated translation skipping site positioned between the TCR α and TCR β chain. Constructs for making P2A containing proteins (also referred to as 2A Peptide-Linked multicistronic vectors) are known in the art. (See, for example, Gene Transfer: Delivery and Expression of DNA and RNA, A Laboratory Manual, (2007), Friedman et al., International Standard Book Number (ISBN) 978-087969765-5, the disclosure of which is hereby incorporated by reference in its entirety. Thus, in one embodiment, a fusion protein of the invention includes a P2A amino acid sequence. In embodiments, a fusion protein of the invention can include a linker sequence between the TCR α and TCR β chains.

In one embodiment, the expression construct that encodes the TCR can also encode additional polynucleotides. The additional polynucleotide can be such that it enables identification of TCR expressing T lymphocytes, such as by encoding a detectable marker, such as a fluorescent or luminescent protein. The additional polynucleotide can be such that it encodes an element that allows for selective elimination of TCR expressing cells, such as thymidine kinase gene. In some embodiments the additional polynucleotides can be such that they facilitate inhibition of expression of endogenously encoded TCRs. In an embodiment, the expression construct that encodes the TCR also encodes a polynucleotide which can facilitate RNAi-mediated down-regulation of one or more endogenous TCRs. For example, see Okamoto S, et al. (2009) Cancer Research, 69:9003-9011, and Okamoto S, et al. (2012). Molecular Therapy-Nucleic Acids, 1, e63, the disclosures of which are hereby incorporated by reference in their entireties. In an embodiment, the expression construct that encodes the TCR can encode an shRNA or an siRNA targeted to an endogenously encoded TCR. In an alternative embodiment, a second, distinct expression construct that encodes the polynucleotide for use in downregulating endogenous TCR production can be used.

The T lymphocyte can also be a T lymphocyte modified to express CAR. CARs are molecules that combine antibody-based specificity for a desired antigen (e.g., a tumor antigen or a viral antigen) with a T cell receptor-activating intracellular domain to generate a chimeric protein that exhibits a specific anti-tumor cellular immune activity. CAR exhibited remarkable anti-tumor effects in patients with chronic leukemia (Porter et al., 201 1, New Engl J Med, 365(8): 725-733; Kalos et al, 201 1, Sci. Tr. Med, 3(95): 95ra73). CAR T lymphocytes based therapy can be used in treating cancer including but not limited to hematologic malignancies and solid tumor. In some embodiments, the present invention provides a T lymphocyte expressing a therapeutic tumor directed CAR, the binding of which to a tumor antigen on a cancerous cell can results in the activation of the T lymphocyte and T cell-mediated death of the cancerous cell, wherein the T lymphocyte has been contacted ex vivo with an agent comprising a prostaglandin pathway agonist, a glucocorticoid, or a combination thereof to improve its homing and engraftment properties.

In some embodiments, the T lymphocyte is genetically modified to stably express a CAR. T lymphocytes expressing a CAR can be referred to herein as CAR T cells, CAR T lymphocytes, or CAR modified T cells. The cell can be genetically modified to stably express an antibody binding domain on its surface, conferring novel antigen specificity that is MHC independent. In some embodiments, the T cell is genetically modified to stably express a CAR that combines an antigen recognition domain of a specific antibody with an intracellular domain of the CD3− zeta chain or FcyRI protein into a single chimeric protein.

The CAR T lymphocytes can recognize tumor antigens through the recombinant CAR. In one embodiment, the tumor antigen has one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD 19, CD20 and CD37 are other candidates for target antigens in B-cell lymphoma.

The type of tumor antigen referred to in the invention can be a tumor-specific antigen (TSA) or a tumor-associated antigen (TAA). A TSA is unique to tumor cells and generally does not occur on other cells in the body. A TAA associated antigen is not unique to a tumor cell and instead is also expressed on a normal cell under conditions that fail to induce a state of immunologic tolerance to the antigen. The expression of the antigen on the tumor can occur under conditions that enable the immune system to respond to the antigen. TAAs can be antigens that are expressed on normal cells during fetal development when the immune system is immature and unable to respond or they can be antigens that are normally present at extremely low levels on normal cells but which are expressed at much higher levels on tumor cells.

Non-limiting examples of TSA or TAA antigens include the following: Differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p1 85erbB2, p 1 80erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4(791Tgp72} alpha-fetoprotem, beta-HCG, BCA225, BTAA, CA 125, CA 15-3\CA 27.29BCAA, CA 195, CA 242, CA-50, CAM43, CD68\I, CO-029, FGF-5, G250, Ga733VEpCAM, HTgp-175, M344, MA-50, MG7-Ag, MOV 18, NB/70K, NY-CO-1, RCAS 1, SDCCAG16, TA-90Mac-2 binding proteiiAcyclophilin C-associated protein, TAAL6, TAG72, TLP, and TPS.

In one embodiment, the T lymphocytes are genetically engineered to express a CAR having an extracellular domain that includes an antigen recognition domain, a transmembrane domain, and a cytoplasmic domain. In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex.

With respect to the cytoplasmic domain, the CAR of the invention can be designed to include the CD28 and/or 41BB signaling domain by itself or be combined with any other desired cytoplasmic domain(s) useful in the context of the CAR of the invention. In one embodiment, the cytoplasmic domain of the CAR can be designed to further include the signaling domain of CD3-zeta. For example, the cytoplasmic domain of the CAR can include but is not limited to CD3-zeta, 41BB and CD28 signaling modules and combinations thereof. Accordingly, the invention provides CAR T cells and methods of their use for adoptive therapy.

In one embodiment, the CAR T cells of the invention can be generated by introducing a lentiviral vector comprising a desired CAR, for example a CAR comprising anti-CD19, CD8a hinge and transmembrane domain, and human 41BB and CD3-zeta signaling domains, into the cells. The modulated CAR T cells of the invention are able to replicate in vivo resulting in long-term persistence that can lead to sustained tumor control.

The T lymphocytes can express a plurality of CARs, which are targeted to a plurality of desired antigens. In one embodiment, the T lymphocytes express a plurality of types of CARs expressed on a cell, where binding of a plurality of types of CARs to their target antigen is required for CAR T cell activation. For example, in one embodiment a T lymphocyte can express a first CAR targeted to a first desired antigen and a second CAR targeted to a second desired antigen. In one embodiment, activation of the CAR T cell only occurs when the first CAR binds the first desired antigen and the second CAR binds to the second desired antigen. The dependence on the binding of a plurality of different CARs can improve the specificity of CAR T cell therapies.

In some embodiments, the T lymphocytes also express an inhibitory CAR where binding of the inhibitory CAR to a normal cell results in inhibition of CAR T cell activity. In one embodiment, the inhibitory CAR is co-expressed in the same T lymphocytes as a therapeutic tumor directed CAR. In one embodiment, the inhibitory CAR has an antigen binding domain that recognizes an antigen associated with a normal, non-cancerous, cell and a cytoplasmic domain. The binding of the inhibitory CAR can result in the death of the CAR T lymphocytes, the inhibition of the signal transduction of the therapeutic tumor directed CAR, or the induction of a signal transduction signal that prevents the modified T lymphocytes from exhibiting its anti-tumor activity.

Compositions and methods of making CAR expressing T lymphocytes are known in the art. See e.g., WO 2012079000; WO2014011987, the disclosures of which are hereby incorporated by reference in their entireties.

In one embodiment the invention relates to administering an isolated population or subpopulation of genetically modified T lymphocytes expressing a CAR for the treatment of a patient having cancer or at risk of having cancer using lymphocyte infusion. The T lymphocytes can be from any source, including but not limited to autologous T lymphocytes and allogeneic T lymphocytes. The allogeneic T lymphocytes used for cell therapy are either complete or partial HLA-match with the patient. In some embodiments, the T lymphocyte is obtained from a patient directly following treatment with a non-cellular based treatment and the T lymphocyte is engineered to express the CAR. Autologous T lymphocyte infusion can be used in the treatment. In some embodiments, autologous PBMCs are collected from a patient in need of treatment and T lymphocytes are activated and expanded using the methods described herein and known in the art, modulated ex vivo with an agent comprising a prostaglandin pathway agonist, a glucocorticoid, or a combination thereof using methods of the present invention to improve its homing and engraftment properties, and then infused back into the patient.

The present invention provides compositions and methods for modulating an isolated population or subpopulation of T lymphocytes with one or more agents under conditions sufficient to improve its therapeutic potential. As described in detail above, the isolated population of T lymphocytes can include any type of T lymphocyte, or mixture of different types of T lymphocytes. The T lymphocytes can be isolated from a living organism, such as human peripheral blood or cord blood. The T lymphocytes can also be differentiated from stem cells or progenitor cells, such as iPSC or ESC. The T lymphocytes can also be genetically modified to have an exogenous polynucleotide, such as a T lymphocyte expressing a recombinant TCR or CAR or any other polynucleotide encoding a therapeutically beneficial polypeptide. Using methods provided in this invention, these T lymphocytes can be modulated ex vivo to have improved properties for cell based therapies. In some embodiments, the modulated T lymphocytes have reduced production of inflammatory cytokine or decreased allogeneic response compared to a noncontacted population or subpopulation of T lymphocytes. In other embodiments, the ex vivo modulation improves the persistence and long term survival of the T lymphocytes. In one embodiment, the modulated T lymphocytes comprise increased percentage or number of Treg cells compared to a noncontacted population or subpopulation of T lymphocytes. In some embodiments, the modulated T lymphocytes have an increased level of one or more Treg effector molecules. In some embodiments, the one or more enhanced Treg effector molecules is selected from the group consisting of FGL2, NR4A2, AREG, TGFB1, CD55, CCR4, THFAIP3, NLRP3, CTLA4, and CXCR4. In some other embodiments, the one or more enhanced Treg effector molecules is selected from the group consisting of Plaur, Fosl2, Ccr8, Areg, Nr4a2, Pdcd1, Nr4a3, and Ctla4. In some embodiments, the modulated T lymphocytes comprise Treg cells having improved survival. In some embodiments, the modulated T lymphocytes comprise Treg cells having increased expression level of genes relating to GvHD attenuation. In some embodiments, the modulated T lymphocytes comprise Treg cells having increased expression level of genes relating to tissue injury prevention. In some embodiments, the modulated T lymphocytes comprise Treg cells having increased expression level of genes relating to CD4+ cell suppression. Described below are exemplary embodiments of the ex vivo modulation of the isolated population or subpopulation of T lymphocytes to improve their therapeutic potential. The conditions include, but are not limited to the agent used and the concentrations thereof, the time the cells are exposed to the agent, and the temperature of treatment.

In some embodiments, the agent can be a prostaglandin pathway agonist, a glucocorticoid, or a combination thereof. A prostaglandin pathway agonist refers to an agent that stimulates prostaglandin cell signaling pathways, including but not limited to an agent that stimulates the PGE2R2 and/or PGE2R4 cell signaling pathways. In some aspects, a prostaglandin pathway agonist is a PGE receptor agonist.

Illustrative examples of prostaglandin pathway agonists that are suitable for use in preparing cells of the invention, include, but are not limited to, PGE₂, dmPGE₂, 15(S)-15-methyl PGE₂, 20-ethyl PGE₂, 8-iso-16-cyclohexyl-tetranor PGE₂, and PGE₂ analogues. In certain embodiments, PGE₂R2 and PGE₂R4 agonists and analogues thereof are of particular interest, and in some embodiments, the agent preferentially binds and activates a PGE₂ EP2 or PGE₂ EP4 receptor.

In some embodiments, the agent is prostaglandin E2 or PGE₂, which includes, without limitation, any naturally occurring or chemically synthesized PGE₂ molecule, as well as analogues or derivatives thereof.

Illustrative examples of PGE₂ analogues and derivatives include, without limitation, 16,16-dimethyl PGE₂ (“dmPGE₂”), 16,16-dimethyl PGE₂ p-(p-acetamidobenzamido) phenyl ester, 1-deoxy-16,16-dimethyl PGE₂, 9-deoxy-9-methylene-16, 16-dimethyl PGE₂, 9-deoxy-9-methylene PGE₂, 9-keto Fluprostenol, 5-trans PGE₂, 17-phenyl-omega-trinor PGE₂, PGE₂ serinol amide, PGE₂ methyl ester, 16-phenyl tetranor PGE₂, 15(S)-15-methyl PGE₂, 15(R)-15-methyl PGE₂, 8-iso-15-keto PGE₂, 8-iso PGE2 isopropyl ester, 8-iso-16-cyclohexyl-tetranor PGE₂, 20-hydroxy PGE₂, 20-ethyl PGE₂, 11-deoxy PGEi, nocloprost, sulprostone, butaprost, 15-keto PGE₂, and 19 (R) hydroxy PGE₂. Also included are PG analogues or derivatives having a similar structure to PGE₂ that are substituted with halogen at the 9-position (see, e.g., WO 2001/12596, the disclosure of which is hereby incorporated by reference in its entirety), as well as 2-decarboxy-2-phosphinico prostaglandin derivatives, such as those described in U.S. Publication No. 2006/0247214, the disclosure of which is hereby incorporated by reference in its entirety).

PGEi analogues, including without limitation alprostadil, can also be used to activate the PGE₂R2 (EP2) and PGE₂P (EP4) cell signaling pathways, and are contemplated as agents useful in the methods of the invention.

Stimulation/activation of the PGE₂R2 (EP2) and PGE₂P (EP4) cell signaling pathways are contemplated to underlie the physiological responses in HSPCs that increase engraftment, maintain cell viability, and increase homing of the cells. Accordingly, in one embodiment, a “non-PGE₂-based ligand” that binds to and stimulates PGE₂R2 and PGE₂P receptors (i.e., a PGE₂R2/PGE₂R4 agonist) is contemplated for use in the methods of the invention.

Illustrative examples of non-PGE₂-based EP₂ receptor agonists include CAY10399, ONO_8815Ly, ONO-AEl-259, CP-533,536 and carbazoles and fluorenes disclosed in WO 2007/071456, the disclosure of which is hereby incorporated by reference in its entirety.

Illustrative examples of non-PGE₂-based EP₄ agonists include ONO-4819, APS-999 Na, AH23848, ONO-AE1-329, and other non-PGE₂-based EP₄ agonists disclosed in WO/2000/038663; U.S. Pat. Nos. 6,747,037; and 6,610,719; the disclosures of which are hereby incorporated by reference in their entireties.

Agents selective for the PGE₂ EP₄ receptor preferentially bind to and activate PGE₂ EP₄ receptors. Such agents have a higher affinity for the EP₄ receptor than for any of the other three EP receptors namely EPi, EP₂ and EP3. Agents that selectively bind the PGE EP₄ receptor include, but are not limited to, agents selected from the group consisting of: 5-[(1E,3R)-4,4-difluoro-3-hydroxy-4-phenyl-1-buten-1-yl]-1-[6-(2H-tetrazol-5R-yl)hexyl]-2-pyrrolidinone; 2-[3-[(1R,2S,3R)-3-hydroxy-2-[(E,3S)-3-hydroxy-5-[2-(methoxymethyl)phenyl]pent-1-enyl]-5-oxocyclopentyljsulfanylpropylsulfanyl] acetic acid; methyl 4-[2-[(1R,2R,3R)-3-hydroxy-2-[(E,3S)-3-hydroxy-4-[3-(methoxymethyl)phenyl]but-1-enyl]-5-oxocyclopentyl]ethyl sulfanyl]butanoate; 16-(3-Methoxymethyl)phenyl-ro-tetranor-5-thiaPGE; 5-{3-[(2S)-2-{(3R)-3-hydroxy-4-[3-(trifluoromethyl)phenyl]butyl}-5-oxopyrrolidin-1-yl]propyl]thiophene-2-carboxylate; [4′-[3-butyl-5-oxo-1-(2-trifluoromethyl-phenyl)-1,5-dihydro-[1,2,4]triazol-4-ylmethyl]-biphenyl-2-sulfonic acid (3-methyl-thiophene-2-carbonyl)-amide]; and ((Z)-7-{(1R,4S,5R)-5-[(E)-5-(3-chloro-benzo[b]thiophene-2-yl)-3-hydroxy-pent-1-enyl]-4-hydroxy-3,3-dimethyl-2-oxo-cyclopentyl}-hept-5-enoic acid), and pharmaceutically acceptable salts of any of these agents.

In particular embodiments, the prostaglandin pathway agonist is PGE₂, dmPGE₂, 15(S)-15-methyl PGE₂, 20-ethyl PGE₂, or 8-iso-16-cyclohexyl-tetranor PGE₂.

In some embodiments, more than one prostaglandin pathway agonist can be used. For example, the agent used for modulating T lymphocytes can be two, three, four, five, or more than five different prostaglandin pathway agonists. A person of ordinary skill in the art would understand the instant invention includes using any combination of the prostaglandin pathway agonists disclosed above or in the references incorporated herein for modulating T lymphocytes.

Illustrative examples of glucocorticoids and glucocorticoid receptor agonists suitable for use in the methods of the invention include, but are not limited to, medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, Cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol, as well as combinations thereof. In particular embodiments, the glucocorticoid includes medrysone, hydrocortisone, triamcinolone, alclometasone, or dexamethasone. In more particular embodiments, the glucocorticoid is dexamethasone.

In some embodiments, more than one glucocorticoids or glucocorticoid receptor agonists can be used. For example, the agent used for modulating T lymphocytes can be two, three, four, five, or more than five different glucocorticoids and glucocorticoid receptor agonists. A person of ordinary skill in the art would understand the instant invention includes using any combination of the glucocorticoids and/or glucocorticoid receptor agonists disclosed above or in the references incorporated herein for modulating T lymphocytes.

Combinations of agents can also be used in preparing the improved population or subpopulation of T lymphocytes of the invention, and in particular embodiments treating population or subpopulation of T lymphocytes with a combination of agents results in an unexpected synergistic effect in reducing T lymphocytes' ability to produce allogeneic response, which correlates to improved therapeutic properties of the treated cells compared to control, vehicle, or non-treated cells.

In some embodiments, an isolated population or subpopulation of T lymphocytes are treated with a combination comprising one or more prostaglandin pathway agonists and one or more glucocorticoids.

In particular embodiments of the invention, the prostaglandin pathway agonist comprised in the combination is a compound that selectively binds the PGE₂ EP₂ or the PGE₂ EP₄ receptor. In other embodiments of the invention, the prostaglandin pathway agonist includes PGE₂, or a PGE₂ analogue or derivative thereof. In particular embodiments, the prostaglandin pathway agonist is PGE₂, dmPGE₂, 15(S)-15-methyl PGE₂, 20-ethyl PGE₂, or 8-iso-16-cyclohexyl-tetranor PGE₂. In more particular embodiments of the invention, the prostaglandin pathway agonist is PGE₂ or 16,16-dimethyl PGE₂.

In some embodiments, the glucocorticoid in the combination is selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, Cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol.

In some aspects, the glucocorticoid comprised in the combination includes medrysone, hydrocortisone, alclometasone, dexamethasone, methylprednisolone, or triamcinolone. In one embodiment, the glucocorticoid is dexamethasone.

In some aspects, the isolated population or subpopulation of T lymphocytes are treated with a combination comprising a prostaglandin pathway agonist selected from the group consisting of PGE₂, dmPGE₂, 15(S)-15-methyl PGE₂, 20-ethyl PGE₂, and 8-iso-16-cyclohexyl-tetranor PGE₂ and one or more glucocorticoids. In a particular embodiment, T lymphocytes are treated with a combination comprising PGE₂ or dmPGE₂ and a glucocorticoid.

In some aspects, the isolated population or subpopulation of T lymphocytes are treated with a combination comprising a prostaglandin pathway agonist selected from the group consisting of PGE₂, dmPGE₂, 15(S)-15-methyl PGE₂, 20-ethyl PGE₂, and 8-iso-16-cyclohexyl-tetranor PGE₂, and a glucocorticoid selected from the group consisting of medrysone, hydrocortisone, alclometasone, dexamethasone, methylprednisolone, or triamcinolone.

In other aspects, the combination includes PGE₂ or dmPGE₂ and medrysone, hydrocortisone, alclometasone, dexamethasone, methylprednisolone, or triamcinolone. In certain embodiments, the T lymphocyte is treated with a combination comprising PGE₂ or dmPGE₂ and dexamethasone. In one embodiment, the T lymphocyte is treated with a combination of dmPGE₂ and dexamethasone.

In some embodiments, a combination of more than one prostaglandin pathway agonists and more than one glucocorticoids or glucocorticoid receptor agonists can be used. In some aspects, the combination of agents can include two, three, four, five, or more than five different prostaglandin pathway agonists and a glucocorticoid and glucocorticoid receptor agonist. In other aspects, the combination of agents can include one prostaglandin pathway agonist and two, three, four, five, or more than five different a glucocorticoids and glucocorticoid receptor agonists. In yet other aspects, the combination of agents can include two, three, four, five, or more than five different prostaglandin pathway and two, three, four, five, or more than five different a glucocorticoids and glucocorticoid receptor agonists.

A person of ordinary skill in the art would understand the instant invention includes using any combination of the prostaglandin pathway agonists and the glucocorticoids or glucocorticoid receptor agonists disclosed above or in the references incorporated herein for ex vivo modulating an isolated population or subpopulation of T lymphocytes to improve their therapeutic potential, including improved engraftment potential and reduced risk to initiate an allogeneic response.

In some embodiments, the isolated population or subpopulation of T lymphocytes are contacted with one or more agents, each at a final concentration of about 1 μM to about 100 μM. In certain embodiments, T lymphocytes are treated with one or more agents, each at a final concentration of about 1×10⁻¹⁴ M to about 1×10⁻³ M, about 1×10⁻¹³ M to about 1×10⁻⁴ M, about 1×10⁻¹² M to about 1×10⁻⁵ M, about 1×10⁻¹¹ M to about 1×10⁻⁴ M, about 1×10⁻¹¹ M to about 1×10⁻⁵ M, about 1×10⁻¹⁰ M to about 1×10⁻⁴ M, about 1×10⁻¹⁰ M to about 1×10⁻⁵ M, about 1×10⁻⁹ M to about 1×10⁻⁴ M, about 1×10⁻⁹ M to about 1×10⁻⁵ M, about 1×10⁻⁸ M to about 1×10⁻⁴ M, about 1×10⁻⁷ M to about 1×10⁻⁴ M, about 1×10⁻⁶ M to about 1×10⁻⁴ M, or any intervening ranges of final concentrations.

In some embodiments, the isolated population or subpopulation of T lymphocytes are treated with one or more agents, each at a final concentration of about 1×10⁻¹⁴ M, about 1×10⁻¹³ M, about 1×10⁻¹² M, about 1×10⁻¹⁰ M, about 1×10⁻⁹ M, about 1×10⁻⁸ M, about 1×10⁻⁷ M to about 1×10⁻⁶ M, about 1×10⁻⁵ M, about 1×10⁻⁴ M, about 1×10⁻³ M, or any intervening final concentration. In treatments comprising one or more agents, the agents can be at different concentrations from each other or at the same concentration.

In some embodiments, the isolated population or subpopulation of T lymphocytes are treated with one or more agents, each at a final concentration of about 10 nM to about 100 μM, about 100 nM, about 500 nM, about 1 μM, about 10 μM, about 20 μM, about 30 μM, about 40 μM, about 50 μM, about 60 μM, about 70 μM, about 80 μM, about 90 μM, about 100 μM, about 110 μM, or about 120 μM, or any other intervening concentration of the agent (e.g., 0.1 μM, 1 μM, 5 μM, 10 μM, 20 μM, 50 μM, 100 μM). In one embodiment, the concentration of each agent is a final concentration of about 10 μM to about 25 μM. In one embodiment, the sufficient concentration of an agent is a final concentration of about 10 μM. In one embodiment, T lymphocytes are treated with 10 μM dmPGE₂ and 10 μM dexamethasone.

In some embodiments, the isolated population or subpopulation of T lymphocytes are treated (e.g., contacted with one or more agents) 1, 2, 3, 4, 5, 6, 7, 8, 9 or 10 or more times. The isolated population or subpopulation of T lymphocytes can be intermittently, episodically, or sequentially contacted with one or more agents within the same vessel (e.g., contacting the population of cells with one drug for a period of time, exchanging the culture medium and/or washing the population of cells, then repeating the cycle with the same or a different combination of pharmaceutical agents for the same predetermined period of time or a different predetermined period of time).

In various embodiments, sufficient temperature conditions include incubation of the isolated population or subpopulation of T lymphocytes with the one or more agents at a physiologically relevant temperature, such as a temperature range of about 22° C. to about 39° C. (about room temperature to about body temperature), including but not limited to temperatures of about 22° C., 23° C., 24° C., 25° C., 26° C., 27° C., 28° C., 29° C., 30° C., 31° C., 32° C., 33° C., 34° C., 35° C., 36° C., 37° C., 38° C., and 39° C. In a particular embodiment, the sufficient temperature condition is between about 35° C. and 39° C. In one embodiment, the sufficient temperature condition is about 37° C.

In various embodiments, the sufficient time period for treating the isolated population or subpopulation of T lymphocytes with one or more agents is an incubation period of about 60 minutes to about 24 hours, about 60 minutes to about twelve hours, about 60 minutes to about 6 hours, about 2 hours to about 6 hours, about 2 hours to about 4 hours, and including, but not limited to, treatment for a duration of about 60 minutes, about 70 minutes, about 80 minutes, about 90 minutes, about 100 minutes, about 110 minutes, about 2 hours, about 2.5 hours, about 3 hours, about 3.5 hours or about 4 hours or any other intervening duration. In a particular embodiment, the sufficient incubation period is about 2 hours to about 4 hours. In one embodiment, the sufficient incubation period for treating the isolated population or subpopulation of T lymphocytes is about 4 hours.

In particular embodiments, the isolated population or subpopulation of T lymphocytes are contacted ex vivo at a temperature range of about 22° C. to about 39° C.; with a prostaglandin pathway agonist at a final concentration of about 10 μM to about 25 μM, and with a glucocorticoid about 10 μM to about 25 M; and incubated with the agents for about 1 hour to about 4 hours, for about 2 hours to about 3 hours, for about 2 hours to about 4 hours, or for about 3 hours to about 4 hours.

In particular embodiments, the isolated population or subpopulation of T lymphocytes are contacted ex vivo at a temperature range of about 22° C. to about 39° C.; at a final concentration of about 10 μM to about 25 μM of PGE₂ or dmPGE₂, and about 10 μM to about 25 M of a glucocorticoid; and incubation with the agents for about 1 hour to about 4 hours, for about 2 hours to about 3 hours, for about 2 hours to about 4 hours, or for about 3 hours to about 4 hours.

In particular embodiments, the isolated population or subpopulation of T lymphocytes is contacted ex vivo at a temperature range of about 22° C. to about 39° C.; at a final concentration of about 10 μM to about 25 μM of PGE₂ or dmPGE₂, and about 10 μM to about 25 μM of a compound selected from the group consisting of medrysone, hydrocortisone, alclometasone, dexamethasone, methylprednisolone, or triamcinolone; and incubation with the agents (compounds) for about 1 hour to about 4 hours, for about 2 hours to about 3 hours, for about 2 hours to about 4 hours, or for about 3 hours to about 4 hours.

In particular embodiments, the isolated population or subpopulation of T lymphocytes are contacted ex vivo at a temperature range of about 22° C. to about 39° C.; at a final concentration of about 10 μM to about 25 μM of a prostaglandin pathway agonist, and about 10 μM to about 25 M of dexamethasone; and incubation with the agents for about 1 hour to about 4 hours, for about 2 hours to about 3 hours, for about 2 hours to about 4 hours, or for about 3 hours to about 4 hours.

In another embodiment, the isolated population or subpopulation of T lymphocytes is contacted ex vivo at a temperature of about 37° C. (about body temperature); a final concentration of about 10 μM PGE₂ or 16,16-dimethyl PGE₂, in combination with a final concentration of about 10 μM of a compound selected from the group consisting of medrysone, hydrocortisone, alclometasone, dexamethasone, methylprednisolone, or triamcinolone; and incubation for about four hours.

In another embodiment, the isolated population or subpopulation of T lymphocytes is contacted ex vivo at a temperature of about 37° C. (about body temperature), with a final concentration of about 10 μM PGE₂ or 16,16-dimethyl PGE₂, in combination with a final concentration of about 10 μM of dexamethasone for about four hours.

Accordingly, a person of ordinary skill in the art would understand that the method of the present invention include using any permutation or combination of the treatment conditions as described herein to improve the therapeutic potential of an isolated population or subpopulation of T lymphocytes. Such treatment conditions include, the agent (or combinations of agent), the concentration of the agent, temperature, and time of incubation.

The modulated population or subpopulation of T lymphocytes of the present invention having improved therapeutic potential can be characterized by having different gene expression profiles. In some embodiments, the different gene expression profiles can indicate that the modulated T lymphocytes are less likely to produce an allogeneic response when transplanted to a patient.

In some embodiments, the isolated population or subpopulation of T lymphocytes is treated with one or more modulating agents in an amount sufficient to increase the expression of one or more signature genes. Such signature genes include, but are not limited to, those selected from the group consisting of: BCL2-like 11(“BCL2L11”; 043521 (UniProtKB Entry No.; same below)); FBJ osteosarcoma oncogene (“FOS”; P01100); Fos-related antigen 2 (“FOSL2”; P15408); myeloid cell leukemia 1 (“MCL1”; Q07820); NLR family pyrin domain containing 3 (“NLRP3”; Q96P20); nuclear receptor subfamily 4, group A, member 3 (“NR4A3”; Q92570); serum/glucocorticoid regulated kinase 1 (“SGK1”; 000141); vascular endothelial growth factor A (“VEGFA”; P15692); cyclin A1 (“CCNA1”; P78396); cyclin D1 (“CCND1”; P24385); cyclin D3 (“CCND3”; P30281); coronin, actin binding protein, 1C (“CORO1C”; Q9ULV4); heparin-binding EGF-like growth factor (“HBEGF”; Q99075); S100 calcium binding protein P (“S100P”; Q96BU1); chemokine (C—C motif) receptor 4 (“CCR4”; P51679); chemokine (C—X—C motif) ligand 2 (“CXCL2”; P19875), chemokine (C—X—C motif) ligand 3 (“CXCL3”; P19876); chemokine (C—X—C motif) ligand 5 (“CXCL5”; P42830); chemokine (C—X—C motif) ligand 6 (“CXCL6”; P80162), CXC chemokine receptor 2 (“CXCR2”; P25025), CCAAT/enhancer binding protein (C/EBP), beta (“CEBPB”; P17676); CCAAT/enhancer binding protein (C/EBP), delta (“CEBPD”; P49716); cytotoxic T-lymphocyte-associated protein 4 (“CTLA4”; P16410), fibrinogen-like protein 2 (“FGL2”; Q14314); FK506 binding protein 5 (“FKBP5”; Q13451); inducible T-cell co-stimulator ligand (“ICOSLG”; 075144); interleukin 21 receptor (“IL21R”; Q9HBE5); melanoma cell adhesion molecule (“MCAM”; P43121); suppressor of cytokine signaling 1(“SOCS1”; 015524); tumor necrosis factor (ligand) superfamily, member 4 (“TNFSF4”; P23510); transducer of ERBB2, 1 (“TOB1”; P50616), and TSC22 domain family, member 3 (“TSC22D3”; Q99576), compared to untreated population or subpopulation of T lymphocytes.

In some embodiments, the isolated population or subpopulation of T lymphocytes is treated with a prostaglandin pathway agonist and a glucocorticoid in an amount sufficient to increase the expression of one or more signature genes selected from the group consisting of: BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, IL21R, MCAM, SOCS1, TNFSF4, TOB1, TSC22D3, FGL2, NR4A2, AREG, TGFB1, CD55, THFAIP3, and CXCR4 compared to an untreated population or subpopulation of T lymphocytes. In some embodiments, the isolated population or subpopulation of T lymphocytes is treated with a prostaglandin pathway agonist and a glucocorticoid in an amount sufficient to increase the expression of one or more Treg effector molecules selected from the group consisting of FGL2, NR4A2, AREG, TGFB1, CD55, CCR4, THFAIP3, NLRP3, CTLA4, and CXCR4, compared to an untreated population or subpopulation of T lymphocytes. In some other embodiments, the one or more enhanced Treg effector molecules is selected from the group consisting of Plaur, Fosl2, Ccr8, Areg, Nr4a2, Pdcd1, Nr4a3, and Ctla4.

In some embodiments, the isolated population or subpopulation of T lymphocytes is treated with dmPGE₂ and dexamethasone in an amount sufficient to increase the expression of one or more signature genes selected from the group consisting of: BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, IL21R, MCAM, SOCS1, TNFSF4, TOB1, TSC22D3, FGL2, NR4A2, AREG, TGFB1, CD55, THFAIP3, and CXCR4 compared to an untreated population or subpopulation of T lymphocytes. In some embodiments, the isolated population or subpopulation of T lymphocytes is treated with with dmPGE₂ and dexamethasone in an amount sufficient to increase the expression of one or more Treg effector molecules selected from the group consisting of FGL2, NR4A2, AREG, TGFB1, CD55, CCR4, THFAIP3, NLRP3, CTLA4, and CXCR4, compared to an untreated population or subpopulation of T lymphocytes. In some other embodiments, the one or more enhanced Treg effector molecules is selected from the group consisting of Plaur, Fosl2, Ccr8, Areg, Nr4a2, Pdcd1, Nr4a3, and Ctla4.

In some embodiments, the isolated population or subpopulation of T lymphocytes is treated with dmPGE₂ and dexamethasone in an amount sufficient to increase the expression of BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, IL21R, MCAM, SOCS1, TNFSF4, TOB1, TSC22D3, FGL2, NR4A2, AREG, TGFB1, CD55, THFAIP3, and CXCR4, compared to an untreated population or subpopulation of T lymphocytes. In some embodiments, the isolated population or subpopulation of T lymphocytes is treated with with dmPGE₂ and dexamethasone in an amount sufficient to increase the expression of FGL2, NR4A2, AREG, TGFB1, CD55, CCR4, THFAIP3, NLRP3, CTLA4, and CXCR4, compared to an untreated population or subpopulation of T lymphocytes.

In some embodiments, the isolated population or subpopulation of T lymphocytes that have been treated with one or more modulating agents have a unique gene expression signature wherein expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or all 38 of the signature genes selected from the group consisting of: BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, L21R, MCAM, SOCS1, TNFSF4, TOB1, TSC22D3, FGL2, NR4A2, AREG, TGFB1, CD55, THFAIP3, and CXCR4 is increased, compared to an untreated population or subpopulation of T lymphocytes. A person skilled in the art would understand that the expression levels of a panel of any combination of the genes listed above can be used to measure the effect of modulation of the isolated population or subpopulation of T lymphocytes. In other words, the ex vivo modulation of an isolated population or subpopulation of T lymphocytes can result in an increase in expression of any combinations of the genes listed above under various circumstances.

In some embodiments, the isolated population or subpopulation of T lymphocytes that have been treated with one or more modulating agents have increased expression of at least one signature gene selected from the group consisting of: activating transcription factor 3 (“ATF3”; P 18847); cAMP responsive element modulator (“CREM”; Q03060); GTP binding protein overexpressed in skeletal muscle (“GEM”; P55040); CXCL2; matrix metallopeptidase 9 (“MMP9”; P14780); plasminogen activator, urokinase receptor (“PLAUR”; Q03405); amphiregulin (“AREG”; P15514); BCL2-related protein A1 (“BCL2A1”; Q16548); dual specificity phosphatase (“DUSP4”; Q13115); FOS; FOSL2; jun proto-oncogene (“JUN”; P05412); myelocytomatosis oncogene (“MYC”; P01106); nuclear receptor subfamily 4, group A, member 2 (“NR4A2”; P43354); NR4A3, SOCS1; suppressor of cytokine signaling 3 (“SOCS3”; 014543); and UL16 binding protein 2 (“ULBP2”), compared to an untreated population or subpopulation of T lymphocytes.

In some embodiments, the isolated population or subpopulation of T lymphocytes is treated with a prostaglandin pathway agonist in an amount sufficient to increase the expression of one or more signature genes selected from the group consisting of: ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2, compared to an untreated population or subpopulation of T lymphocytes.

In some embodiments, the isolated population or subpopulation of T lymphocytes is treated with dmPGE2 in an amount sufficient to increase the expression of one or more signature genes selected from the group consisting of: ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2, compared to an untreated population or subpopulation of T lymphocytes.

In some embodiments, the isolated population or subpopulation of T lymphocytes is treated with dmPGE2 in an amount sufficient to increase the expression of ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2, compared to an untreated population or subpopulation of T lymphocytes.

In other embodiments, the isolated population or subpopulation of T lymphocytes that have been treated with one or more modulating agents as disclosed herein can have a unique gene expression signature comprising expression of 1, 2, 3, 4, 5, 6, 7, 8, 9, 10, 11, 12, 13, 14, 15, 16, 17 or 18 of the signature genes selected from the group consisting of: ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2 is increased, compared to an untreated population or subpopulation of T lymphocytes. A person skilled in the art would understand that the expression levels of a panel of any combination of the genes listed above can be used to measure the effect of modulation of the isolated population or subpopulation T lymphocytes. In other words, the ex vivo modulation of an isolated population or subpopulation of T lymphocytes can result in an increase in expression of any combinations of the genes listed above.

In some embodiments, the modulated population or subpopulation of T lymphocytes have a gene expression signature, wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more of the signature genes disclosed herein is increased by at least 1.1, 1.2, 1.3, 1.4, 1.5, 2, 3, 4, 5, 6, 7, 8, 9, or 10 fold compared to an untreated population or subpopulation of T lymphocytes. A person of ordinary skill in the art would understand that, when more than one signature gene is involved, the expression levels of different genes can differ, with some genes having a higher increase than the others. For example, the expression of two signature genes can be increased, wherein one gene is increased by at least 2 fold, and the expression of the other genes is increased by at least 3 fold. In some embodiments, the average increase of all signature genes is at least about 3 fold. In some embodiments, the average increase of all signature genes is at least about 4 fold.

In some embodiments, the treated population or subpopulation of T lymphocytes has increased expression of PD-1 when compared to an untreated population or subpopulation of T lymphocytes. In some aspects, the treated population or subpopulation of T lymphocytes has increased expression of PD-1 at the cell surface of the T lymphocytes when compared to an untreated population or subpopulation of isolated T lymphocytes. In some aspects, the PD-1 expression in the treated population or subpopulation of T lymphocytes can be increased by at least 10%, 20%, 30%, 40%, 50%, 60%, 70%, 80%, 90%, 100%, 150%, 200%, 250%, 300%, 400%, or 500% compared to an untreated population or subpopulation of T lymphocytes. In other aspects, the PD-1 expression in a treated population or subpopulation of T lymphocytes can be increased by about 10%, 20%, 30%, 40%, 50%, 60%, 70%, 80%, 90%, 100%, 150%, 200%, 250%, 300%, 400%, or 500% compared to an untreated population or subpopulation of T lymphocytes. In some embodiments, the PD-1 expression at the cell surface of the treated population or subpopulation of T lymphocytes is increased by about 20% compared to an untreated population or subpopulation of T lymphocytes. In other embodiments, the PD-1 expression at the cell surface of the treated population or subpopulation of T lymphocytes is increased by about 50% compared to an untreated population or subpopulation of T lymphocytes.

In some embodiments, the treated population or subpopulation of T lymphocytes has decreased expression of ICOS. In some embodiments, the treated population or subpopulation of T lymphocytes has decreased expression of ICOS at the cell surface. In some embodiments, the treated population or subpopulation of T lymphocytes has decreased expression of 41BB. In some embodiments, the treated population or subpopulation of T lymphocytes has decreased expression of 41BB at the cell surface. In some aspects, the ICOS expression or 41BB expression of the treated population or subpopulation of T lymphocytes can be decreased by at least 5%, 10%, 20%, 30%, 40%, 50%, 60%, 70%, 80%, 90%, or 100% compared to an untreated population or subpopulation of T lymphocytes. In some aspects, the ICOS expression or 41BB expression of the treated population or subpopulation of T lymphocytes can be decreased by about 5%, 10%, 20%, 30%, 40%, 50%, 60%, 70%, 80%, 90%, or 100% compared to an untreated population or subpopulation of T lymphocytes. In some embodiments, the expression of ICOS at cell surface of the treated population or subpopulation of T lymphocytes is decreased by about 25%, or by about 35% compared to an untreated population or subpopulation of T lymphocytes. In some embodiments, the expression of 41BB at cell surface of the population or subpopulation of T lymphocytes is decreased by about 25% compared to an untreated population or subpopulation of T lymphocytes. A person of ordinary skill in the art would understand that the modulated population or subpopulation of T lymphocytes can have any permutation and combination of changes in expression of the different genes as described herein.

The gene expression or gene expression signature of the treated population or subpopulation of T lymphocytes can be determined after cells are treated with an agent, or cells can be incubated for some period of time after treatment before determining the gene expression signature of the cells. For example, cells can be treated ex vivo with one or more agents, washed to remove the agents, and the gene expression analyzed without further incubation of the cells. Alternatively, cells can be treated with one or more agents, washed to remove the agents from the cell population, and then the cells are incubated ex vivo for some period of time prior to analyzing the gene expression signature of the cells.

A sample for measurement of the genes disclosed herein can include a heterogeneous or homogenous population of cells and the cell populations can be purified or not purified from the sample. The expression of a gene, such as FOS, can be measured at the level of cDNA, RNA, mRNA, or combinations thereof. Any methods available in the art for detecting expression of gene are encompassed herein, including such as determining the quantity or presence of an RNA transcript or its expression product of a gene. Methods for detecting expression of genes include methods based on PCR, hybridization analysis of polynucleotides, methods based on sequencing of polynucleotides, immunohistochemistry methods, and proteomics-based methods. The methods generally detect expression products (e.g., mRNA) of the genes of interest. In some embodiments, PCR-based methods, such as reverse transcription PCR (RT-PCR) (Weis et al., TIG 8:263-64, 1992), and array-based methods such as microarray (Schena et al., Science 270:467-70, 1995) are used. The disclosures of the cited references are hereby incorporated by reference in their entireties.

General methods for RNA extraction are well known in the art and are disclosed in standard textbooks of molecular biology, including Ausubel et al., ed., Current Protocols in Molecular Biology, John Wiley & Sons, New York 1987-1999, the disclosure of which is hereby incorporated by reference in its entirety. In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to an intrinsic gene of the present invention, or any derivative DNA or RNA. Hybridization of an mRNA with the probe indicates that the intrinsic gene in question is being expressed.

An alternative method for determining the level of gene expression in a sample involves the process of nucleic acid amplification, for example, by RT-PCR (U.S. Pat. No. 4,683,202), ligase chain reaction (Barany, Proc. Natl. Acad. Sci. USA 88: 189-93, 1991), self sustained sequence replication (Guatelli et al., Proc. Natl. Acad. Sci. USA 87: 1874-78, 1990), transcriptional amplification system (Kwoh et al., Proc. Natl. Acad. Sci. USA 86: 1173-77, 1989), Q-Beta Replicase (Lizardi et al., Bio/Technology 6: 1197, 1988), rolling circle replication (U.S. Pat. No. 5,854,033), or any other nucleic acid amplification method, followed by the detection of the amplified molecules using techniques well known to those of skill in the art.

In particular aspects of the invention, gene expression is assessed by quantitative RT-PCR. Numerous different PCR or QPCR protocols are known in the art and exemplified herein below and can be directly applied or adapted for use using the presently-described compositions for the detection and/or quantification of the signature gene. Quantitative PCR (QPCR) (also referred as real-time PCR) is preferred under some circumstances because it provides not only a quantitative measurement, but also reduced time and contamination. In some instances, the availability of full gene expression profiling techniques is limited due to requirements for fresh frozen tissue and specialized laboratory equipment, making the routine use of such technologies difficult in a clinical setting. The quantitative PCR (or real-time QPCR) direct monitors the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or threshold level of fluorescence varies directly with the concentration of amplifiable targets at the beginning of the PCR process, enabling a measure of signal intensity to provide a measure of the amount of target nucleic acid in a sample in real time.

Normalization can be used to remove sample-to-sample variation. For microarray data, the process of normalization aims to remove systematic errors by balancing the fluorescence intensities of the two labeling dyes. The dye bias can come from various sources including differences in dye labeling efficiencies, heat and light sensitivities, as well as scanner settings for scanning two channels. Some commonly used methods for calculating normalization factor include: (i) global normalization that uses all genes on the array, such as by log scale robust multi-array analysis (RMA); (ii) housekeeping genes normalization that uses constantly expressed housekeeping/invariant genes; and (iii) internal controls normalization that uses known amount of exogenous control genes added during hybridization (Quackenbush (2002) Nat. Genet. 32 (SuppL), 496-501). In one embodiment, expression of the genes disclosed herein can be determined by normalizing the expression to control housekeeping gene expression or by performing log scale robust multi-array analysis (RMA).

In some embodiments, the treated population or subpopulation of T lymphocytes has decreased production of effector cytokine compared to an untreated population or subpopulation of T lymphocytes. In some aspects, the treated population or subpopulation of T lymphocytes has decreased production of effector cytokine compared to an untreated population or subpopulation of T lymphocytes. In some embodiments, the effector cytokine production of the treated population or subpopulation of T lymphocytes is decreased by at least 5%, 10%, 20%, 30%, 40%, 50%, 60%, 70%, 80%, 90%, or 100% compared to an untreated population or subpopulation of T lymphocytes. In some embodiments, the effector cytokine production of the treated population or subpopulation of T lymphocytes is decreased by about 5%, 10%, 20%, 30%, 40%, 50%, 60%, 70%, 80%, 90%, or 100% compared to an untreated population or subpopulation of T lymphocytes. In some aspects, the treated population or subpopulation of T lymphocytes has reduced production of interferon gamma compared to an untreated population or subpopulation of T lymphocytes. In some aspects, the interferon gamma and/or IL17 production of the treated population or subpopulation of T lymphocytes is decreased by at least 5%, 10%, 20%, 30%, 40%, 50%, 60%, 70%, 80%, 90%, or 100% compared to an untreated population or subpopulation of T lymphocytes. In some aspects, the interferon gamma and/or IL17 production of the treated population or subpopulation of T lymphocytes is decreased by about 5%, 10%, 20%, 30%, 40%, 50%, 60%, 70%, 80%, 90%, or 100% compared to an untreated population or subpopulation of T lymphocytes. In one aspect, the interferon gamma production of the treated population or subpopulation of T lymphocytes is decreased by about 50% compared to an untreated population or subpopulation of T lymphocytes. In some embodiments, the treated population or subpopulation of T lymphocytes comprise Treg cells. In some further embodiments, the treated population or subpopulation of T lymphocytes comprise CD4+CD25^{hi} CD127^{lo}Foxp3⁺ cells.

In some embodiments, the treated population or subpopulation of T lymphocytes has increased production of less inflammatory cytokine or anti-inflammatory cytokine compared to an untreated population or subpopulation of T lymphocytes. In some aspects, the production of less inflammatory cytokine or anti-inflammatory cytokine of the modulated population or subpopulation of T lymphocytes can be increased by at least 10%, 20%, 30%, 40%, 50%, 60%, 70%, 80%, 90%, 100%, 150%, 200%, 250%, 300%, 400%, or 500% compared to an untreated population or subpopulation of T lymphocytes. In some embodiments, the treated population or subpopulation of T lymphocytes has increased production of interleukin 4, interleukin 10, or both when compared to an untreated population or subpopulation of T lymphocytes. In some embodiments, the treated population or subpopulation of T lymphocytes comprise Treg cells. In some further embodiments, the treated population or subpopulation of T lymphocytes comprise CD4+CD25^{hi} CD127^{lo}Foxp3⁺ cells.

In one embodiment, the treated population or subpopulation of T lymphocytes has reduced production of interferon gamma and increased production of interleukin 4 or interleukin 10 compared to an untreated population or subpopulation of T lymphocytes. In one embodiment, the treated population or subpopulation of T lymphocytes has reduced production of interferon gamma and increased production of interleukin 4 and interleukin 10 compared to an untreated population or subpopulation of T lymphocytes. A person of ordinary skill in the art would understand that the treated population or subpopulation of T lymphocytes can have any permutation and combination of changes in producing different cytokines as described herein. In some embodiments, the treated population or subpopulation of T lymphocytes comprise Treg cells. In some further embodiments, the treated population or subpopulation of T lymphocytes comprise CD4+CD25^{hi} CD127^{lo}Foxp3⁺ cells. The person of ordinary skill in the art would further understand that the treated population or subpopulation of T lymphocytes can have any combination of changes in gene expression and changes in cytokine production as described herein.

In some embodiments, the present invention provides a pharmaceutical composition having an isolated population or subpopulation of treated T lymphocytes. As described above, the isolated population or subpopulation of modulated T lymphocytes can have enhanced therapeutic potentials. The pharmaceutical composition having the isolated population or subpopulation of treated T lymphocytes can have improved success rate, or reduced relapse rate when used in a cell therapy or a T lymphocytes therapy. For example, the pharmaceutical composition having the isolated population or subpopulation of treated T lymphocytes can have decreased the risk for producing an allogeneic response and increased persistence in the host. The pharmaceutical composition having the isolated population or subpopulation of treated T lymphocytes can produce better long term effects when used in a cell therapy or T cell therapy because the ex vivo treatment can alter the ratios of different types the T lymphocytes in the immune reconstitution. For example, the treatment can increase the ratio of naive T lymphocytes, central memory T cells, or both, in the immune reconstitution. In some embodiments, the treatment can increase the ratio and number of Treg cells in the T lymphocytes population. In some further embodiments, the treatment can increase the ratio and number of Treg cells comprising CD4+CD25^{hi} CD127^{lo}Foxp3⁺ cells.

The isolated population or subpopulation of treated T lymphocytes can have at least 50%, 60%, 70%, 80%, 90%, 95%, 98%, or 99% T lymphocytes. In some embodiments, the treated T lymphocytes comprise Treg cells. In some embodiments the treated T lymphocytes comprise CD4+CD25^{hi} CD127^{lo}Foxp3⁺ cells. In some embodiments, the isolated population or subpopulation is about 95% to about 100% T lymphocytes. In some embodiments, the present invention provides pharmaceutical compositions having purified T lymphocytes, such as a composition having an isolated population of about 95% T lymphocytes to treat a subject in need of the cell therapy. As described above, the isolated population or subpopulation of T lymphocytes can have any type of T lymphocytes, or any combination of two or more types of T lymphocytes. The T lymphocytes can be isolated from a mammal, such as a human, differentiated from a stem or progenitor cell, or recombinantly produced.

In some embodiments, the pharmaceutical composition includes an isolated population or subpopulation of T lymphocytes, wherein the isolated population or subpopulation of T lymphocytes has less than about 0.1%, 0.5%, 1%, 2%, 5%, 10%, 15%, 20%, 25%, or 30% T lymphocytes. In some embodiments, the treated T lymphocytes comprise Treg cells. In some embodiments the treated T lymphocytes comprise CD4+CD25^{hi} CD127^{lo}Foxp3⁺ cells. The isolated population or subpopulation of T lymphocytes in some embodiments can have more than about 0.1%, 0.5%, 1%, 2%, 5%, 10%, 15%, 20%, 25%, or 30% T lymphocytes. In other embodiments, the isolated population or subpopulation of T lymphocytes can have about 0.1% to about 1%, about 1% to about 3%, about 3% to about 5%, about 10%-about 15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% T lymphocytes.

In particular embodiments, the isolated population or subpopulation of T lymphocytes can have about 0.1%, about 1%, about 3%, about 5%, about 10%, about 15%, about 20%, about 25%, about 30%, about 35%, about 40%, about 45%, about 50%, about 60%, about 70%, about 80%, about 90%, about 95%, about 98%, about 99%, or about 100% T lymphocytes.

As a person of ordinary skill in the art would understand, both autologous and allogeneic T lymphocytes can be used in cell therapies or T cell therapies. Autologous cell therapies or T cell therapies can have reduced infection, low probability for GvHD, and rapid immune reconstitution. Allogeneic cell therapies or T cell therapies can have an immune mediated graft-versus-malignancy (GVM) effect, and low rate of relapse. Allogeneic cell therapies or T cell therapies can be used when the autologous T lymphocyte therapy is not available. Based on the specific conditions of the patients or subject in need of the cell therapy or T cell therapy, a person of ordinary skill in the art would be able to determine which specific type of therapy to administer.

In particular embodiments, the isolated population or subpopulation of T lymphocytes of the pharmaceutical composition of the invention are allogeneic to a subject. In particular embodiments, the T lymphocytes of the pharmaceutical formulation of the invention are autologous to a subject. For autologous transplantation, the isolated population or subpopulation of T lymphocytes are either complete or partial HLA-match with the patient. In another embodiment, the T lymphocytes are not HLA-matched to the subject.

In some embodiments, the number of treated T lymphocytes in the pharmaceutical composition is at least 0.1×10⁵ cells, at least 0.5×10⁵ cells, at least 1×10⁵ cells, at least 5×10⁵ cells, at least 10×10⁵ cells, at least 0.5×10⁶ cells, at least 0.75×10⁶ cells, at least 1×10⁶ cells, at least 1.25×10⁶ cells, at least 1.5×10⁶ cells, at least 1.75×10⁶ cells, at least 2×10⁶ cells, at least 2.5×10⁶ cells, at least 3×10⁶ cells, at least 4×10⁶ cells, at least 5×10⁶ cells, at least 10×10⁶ cells, at least 15×10⁶ cells, at least 20×10⁶ cells, at least 25×10⁶ cells, or at least 30×10⁶ cells. In some embodiments, the treated T lymphocytes comprise Treg cells. In some embodiments the treated T lymphocytes comprise CD4+CD25^{hi} CD127^{lo}Foxp3⁺ cells.

In some embodiments, the number of treated T lymphocytes in the pharmaceutical composition is about 0.1×10⁵ cells to about 10×10⁵ cells; about 0.5×10⁶ cells to about 5×10⁶ cells; about 1×10⁶ cells to about 3×10⁶ cells; about 1.5×10⁶ cells to about 2.5×10⁶ cells; or about 2×10⁶ cells to about 2.5×10⁶ cells. In some embodiments, the treated T lymphocytes comprise Treg cells. In some embodiments the treated T lymphocytes comprise CD4+CD25^{hi}CD127^{lo}Foxp3⁺ cells.

In some embodiments, the number of treated T lymphocytes in the pharmaceutical composition is about 1×10⁶ cells to about 3×10⁶ cells; about 1.0×10⁶ cells to about 5×10⁶ cells; about 1.0×10⁶ cells to about 10×10⁶ cells, about 10×10⁶ cells to about 20×10⁶ cells, about 10×10⁶ cells to about 30×10⁶ cells, or about 20×10⁶ cells to about 30×10⁶ cells. In some embodiments, the treated T lymphocytes comprise Treg cells. In some embodiments the treated T lymphocytes comprise CD4+CD25^{hi} CD127^{lo}Foxp3⁺ cells.

In some other embodiments, the number of treated T lymphocytes in the pharmaceutical composition is about 1×10⁶ cells to about 30×10⁶ cells; about 1.0×10⁶ cells to about 20×10⁶ cells; about 1.0×10⁶ cells to about 10×10⁶ cells, about 2.0×10⁶ cells to about 30×10⁶ cells, about 2.0×10⁶ cells to about 20×10⁶ cells, or about 2.0×10⁶ cells to about 10×10⁶ cells. In some embodiments, the treated T lymphocytes comprise Treg cells. In some embodiments the treated T lymphocytes comprise CD4+CD25^{hi} CD127^{lo}Foxp3⁺ cells.

In yet other embodiments, the number of treated T lymphocytes in the pharmaceutical composition is about 1×10⁶ cells, about 2×10⁶ cells, about 5×10⁶ cells, about 7×10⁶ cells, about 10×10⁶ cells, about 15×10⁶ cells, about 17×10⁶ cells, about 20×10⁶ cells about 25×10⁶ cells, or about 30×10⁶ cells. In some embodiments, the treated T lymphocytes comprise Treg cells. In some embodiments the treated T lymphocytes comprise CD4+CD25^{hi} CD127^{lo}Foxp3⁺ cells.

In one embodiment, the number of treated T lymphocytes in the pharmaceutical composition is the number of T lymphocytes in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.5×10⁶ cells/kg of bodyweight, at least 0.75×10⁶ cells/kg of bodyweight, at least 1×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, or at least 30×10⁶ cells/kg of bodyweight. In some embodiments, the treated T lymphocytes comprise Treg cells. In some embodiments the treated T lymphocytes comprise CD4+CD25^{hi} CD127^{lo}Foxp3⁺ cells.

The modulated population or subpopulation of T lymphocytes with improved properties provided by the invention can be administered to a subject without being expanded ex vivo or in vitro. In particular embodiments, an isolated population or subpopulation of T lymphocytes is obtained and treated ex vivo in accordance with the methods provided herein to obtain improved population or subpopulation of T lymphocytes, the improved population or subpopulation of T lymphocytes can be washed to remove the treatment agent(s), and the improved population or subpopulation of T lymphocytes are administered to a patient without expansion of the isolated population or subpopulation of T lymphocytes ex vivo. In some embodiments, the improved population or subpopulation of T lymphocytes are obtained from a donor source, including cord blood, or mobilized peripheral blood and are not expanded prior to or after treatment of the isolated population or subpopulation of T lymphocytes, or at any time prior to administration of the therapeutic composition to a patient. In some embodiments, the modulated T lymphocytes comprise Treg cells. In some embodiments the modulated T lymphocytes comprise CD4+CD25h CD127^{lo}Foxp3⁺ cells.

In one embodiment, an unexpanded population or subpopulation of T lymphocytes is treated and is administered to a patient prior to any substantial ex vivo cell division of the T lymphocytes in the population, or prior to the time required for any substantial cell division ex vivo. In other embodiments, an unexpanded population or subpopulation of T lymphocytes is treated and is administered to a patient prior to any substantial ex vivo mitosis of the T lymphocytes in the isolated population or subpopulation, or prior to the time required for any substantial mitosis ex vivo. In some embodiments, an unexpanded population or subpopulation of T lymphocytes is treated and is administered to a patient prior to the doubling time of the T lymphocytes in the population. In some embodiments, an unexpanded population or subpopulation of T lymphocytes is treated and is administered to a patient within 6, 12, or 24 hours of treatment of the isolated population or subpopulation of T lymphocytes. In other embodiments, an unexpanded population or subpopulation of T lymphocytes is treated and is administered to a patient within 2 hours of treatment of the isolated population or subpopulation of T lymphocytes. In some embodiments, the treated T lymphocytes comprise Treg cells. In some embodiments the treated T lymphocytes comprise CD4+CD25^{hi} CD127^{lo}Foxp3⁺ cells.

In various embodiments, the isolated population or subpopulation of T lymphocytes of the invention are not cultured prior to treatment with one or more agents, or combinations of agents, ex vivo or at any time prior to administration to a patient. In some embodiments, the isolated population or subpopulation of T lymphocytes are cultured for less than about 24 hours prior to treatment with one or more agents. In other embodiments, the isolated population or subpopulation of T lymphocytes are cultured for less than about 12 hours, about 10 hours, about 8 hours, about 6 hours, about 4 hours, or about two hours prior to treatment with one or more agents.

In other embodiments, the invention provides an isolated population or subpopulation of T lymphocytes that are expanded prior to treatment of the isolated population or subpopulation of T lymphocytes with one or more agents. The isolated population or subpopulation of T lymphocytes, whether obtained from cord blood, bone marrow, peripheral blood, Wharton's jelly, placental blood or other source, differentiated from iPSC or ESC, or other stem or progenitor cells, or recombinantly produced to express TCR, CAR or other proteins, can be grown or expanded in any suitable, commercially available or custom defined medium, with or without serum, as desired (see, e.g., Hartshorn et ah, Cell Technology for Cell Products, pages 221-224, R. Smith, Editor; Springer Netherlands, 2007, the disclosure of which is hereby incorporated by reference in its entirety). In some embodiments, autologous PBMCs are collected from a patient in need of treatment and T lymphocytes are activated and expanded using the methods described herein and known in the art, modified with the methods of the invention to improve their therapeutic potential, and then infused back into the patient.

For genetically engineered T lymphocytes that express recombinant TCR or CAR, whether prior to or after genetic modification of the T lymphocytes, the T lymphocytes can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7, 172,869; 7,232,566; 7, 175,843; 5,883,223; 6,905,874; 6,797,514; 6,867,041; and U.S. Patent Application Publication No. 20060121005, the disclosures of which are hereby incorporated by reference in their entireties.

For example, the isolated population or subpopulation of T lymphocytes of the invention can be expanded by contact with a surface having attached thereto an agent that stimulates a CD3 TCR complex associated signal and a ligand that stimulates a co-stimulatory molecule on the surface of the T lymphocytes. In particular, the isolated population or subpopulation of T lymphocytes can be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T lymphocytes, a ligand that binds the accessory molecule is used. For example, an isolated population or subpopulation of T lymphocytes can be contacted with an anti-CD3 antibody and an anti-CD28 antibody, under conditions appropriate for stimulating proliferation of the T lymphocytes. In some embodiments, a population of CD4+ T cells and/or CD8+ T cells are contacted with an anti-CD3 antibody and an anti-CD28 antibody. Examples of an anti-CD28 antibody include, but are not limited to, 9.3, B-T3, XR-CD28 (Diacione, Besancon, France) can be used as can other methods commonly known in the art (Berg et al., Transplant Proc. 30(8):3975-3977, 1998; Haanen et al., J. Exp. Med. 190(9): 13191328, 1999; Garland et al., J. Immunol Meth. 227(1-2):53-63, 1999, the disclosures of which are hereby incorporated by reference in their entireties).

In certain embodiments, the primary stimulatory signal and the co-stimulatory signal for the T cell can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent which will bind to the agents such as disclosed in U.S. Patent Application Publication Nos. 20040101519 and 20060034810 for artificial antigen presenting cells (aAPCs) that are contemplated for use in activating and expanding T lymphocytes in the present invention.

In various embodiments, the pharmaceutical composition having improved T lymphocytes administered to a subject are a heterogeneous population of cells including, whole bone marrow, umbilical cord blood, mobilized peripheral blood, hematopoietic stem cells, hematopoietic progenitor cells, and the progeny of hematopoietic stem and progenitor cells, including granulocytes (e.g., promyelocytes, myelocytes, metamyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages).

The compositions comprising a modulated population or subpopulation of T lymphocytes of the invention can be sterile, and can be suitable and ready for administration (i.e., can be administered without any further processing) to human patients. In some embodiments, the therapeutic composition is ready for infusion into a patient. A cell based composition that is ready for administration means that the composition does not require any further treatment or manipulations prior to transplant or administration to a subject. In some embodiments, the modulated T lymphocytes comprise Treg cells. In some embodiments the modulated T lymphocytes comprise CD4+CD25^{hi} CD127^{lo}Foxp3⁺ cells.

The sterile, therapeutically acceptable compositions suitable for administration to a patient can include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions of the present invention (see, e.g., Remington's Pharmaceutical Sciences, 17^{th} ed. 1985, the disclosure of which is hereby incorporated by reference in it entirety).

In particular embodiments, therapeutic cell compositions having an isolated population or subpopulation of T lymphocytes also have a pharmaceutically acceptable cell culture medium. A therapeutic composition comprising a population of T lymphocytes as disclosed herein can be administered separately by enteral or parenteral administration methods or in combination with other suitable compounds to effect the desired treatment goals. In some embodiments, the T lymphocytes of the therapeutic composition comprise Treg cells. In some embodiments the T lymphocytes of the therapeutic composition comprise CD4+CD25^{hi} CD127^{lo}Foxp3⁺ cells.

The pharmaceutically acceptable carrier and/or diluent must be of sufficiently high purity and of sufficiently low toxicity to render it suitable for administration to the human subject being treated. It further should maintain or increase the stability of the therapeutic composition. The pharmaceutically acceptable carrier can be liquid or solid and is selected, with the planned manner of administration in mind, to provide for the desired bulk, consistency, etc., when combined with other components of the therapeutic composition of the invention. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions of the present invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Such carrier solutions also can contain buffers, diluents and other suitable additives. A buffer refers to a solution or liquid whose chemical makeup neutralizes acids or bases without a significant change in PH. Examples of buffers envisioned by the invention include, but are not limited to, Dulbecco's phosphate buffered saline (PBS), Ringer's solution, 5% dextrose in water (D5W), normal/physiologic saline (0.9% NaCl).

These pharmaceutically acceptable carriers and/or diluents can be present in amounts sufficient to maintain a PH of the therapeutic composition of between about 3 and about 10. As such, the buffering agent can be as much as about 5% on a weight to weight basis of the total composition. Electrolytes such as, but not limited to, sodium chloride and potassium chloride can also be included in the therapeutic composition. In one aspect, the PH of the therapeutic composition is in the range from about 4 to about 10. Alternatively, the PH of the therapeutic composition is in the range from about 5 to about 9, from about 6 to about 9, or from about 6.5 to about 8. In another embodiment, the therapeutic composition includes a buffer having a PH in one of said PH ranges. In another embodiment, the therapeutic composition has a PH of about 7. Alternatively, the therapeutic composition has a PH in a range from about 6.8 to about 7.4. In still another embodiment, the therapeutic composition has a PH of about 7.4.

The sterile composition of the invention can be a sterile solution or suspension in a nontoxic pharmaceutically acceptable medium. Suspension can refer to non-adherent conditions in which cells are not attached to a solid support. For example, cells maintained in suspension can be stirred and are not adhered to a support, such as a culture dish.

A suspension is a dispersion (mixture) in which a finely-divided species is combined with another species, with the former being so finely divided and mixed that it doesn't rapidly settle out. A suspension can be prepared using a vehicle such as a liquid medium, including a solution. In some embodiments, the therapeutic composition of the invention is a suspension, where the stem and/or progenitor cells are dispersed within an acceptable liquid medium or solution, e.g., saline or serum-free medium, and are not attached to a solid support. In everyday life, the most common suspensions are those of solids in liquid water. Among the acceptable diluents, e.g., vehicles and solvents, that can be employed are water, Ringer's solution, isotonic sodium chloride (saline) solution, and serum-free cell culture medium. In some embodiments, hypertonic solutions are employed in making suspensions. In addition, sterile, fixed oils are conventionally employed as a solvent or suspending medium. For parenteral application, particularly suitable vehicles consist of solutions, preferably oily or aqueous solutions, as well as suspensions, emulsions, or implants. Aqueous suspensions can contain substances which increase the viscosity of the suspension and include, for example, sodium carboxymethyl cellulose, sorbitol and/or dextran. In some embodiments, the infusion solution is isotonic to subject tissues. In some embodiments, the infusion solution is hypertonic to subject tissues.

The pharmaceutically acceptable carrier, diluents, and other components comprising the administration-ready pharmaceutical composition of the invention are derived from U.S. Pharmaceutical grade reagents that will permit the therapeutic composition to be used in clinical regimens. Typically, these finished reagents, including any medium, solution, or other pharmaceutically acceptable carriers and/or diluents, are sterilized in a manner conventional in the art, such as filter sterilized, and are tested for various undesired contaminants, such as mycoplasma, endotoxin, or virus contamination, prior to use. The pharmaceutically acceptable carrier in one embodiment is substantially free of natural proteins of human or animal origin, and suitable for storing the population of cells of the pharmaceutical composition, including hematopoietic stem and progenitor cells. The pharmaceutical composition is intended to be administered into a human patient, and thus is substantially free of cell culture components such as bovine serum albumin, horse serum, and fetal bovine serum.

The invention also provides, in part, the use of a pharmaceutically acceptable cell culture medium in particular compositions and/or cultures of the present invention. Such compositions are suitable for administration to human subjects. Generally speaking, any medium that supports the maintenance, growth, and/or health of the T lymphocytes of the invention are suitable for use as a pharmaceutical cell culture medium. In particular embodiments, the pharmaceutically acceptable cell culture medium is a serum free medium.

The pharmaceutical composition can have serum-free medium suitable for storing the modulated isolated population or subpopulation of T lymphocytes. In various embodiments, the serum-free medium is animal-free, and can optionally be protein-free. Optionally, the medium can contain biopharmaceutically acceptable recombinant proteins. Animal-free medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein.

The serum-free medium employed in the present invention is a formulation suitable for use in human therapeutic protocols and products. One serum-free media is QBSF-60 (Quality Biological, Inc.), as described in U.S. Pat. No. 5,945,337. QBSF-60 isoptimized with U.S. Pharmaceutical grade components and is composed of the basal medium IMDM plus 2 mM L-glutamine, 100 U/ml penicillin, 100 μg/ml streptomycin, human injectable grade serum albumin (4 mg/ml) (Alpha Therapeutic Corporation), partially iron saturated human transferrin (300 μg/ml) (Sigma Chemical Corporation or Bayer Corporation) and human recombinant sodium insulin (0.48 U/ml) (Sigma). Other serum-free media known in the art include, but are not limited to: Life Technologies Catalogue StemPro-34 serum free culture media; Capmany, et al., Short-term, serum-free, static culture of cord blood-derived CD34⁺ cells: effects of FLT3-L and MIP-I on in vitro expansion of hematopoietic progenitor cells, Haematologica 84:675-682 (1999); Daley, J P, et al., Ex vivo expansion of human hematopoietic progenitor cells in serum-free StemPro™-34 Medium, Focus 18(3):62-67; Life Technologies Catalogue information on AIM V serum free culture media; BioWhittaker Catalogue information on X-VIVO 10 serum free culture media; U.S. Pat. No. 5,397,706 entitled Serum-free basal and culture medium for hematopoietic and leukemia cells; no cell proliferation; Kurtzberg et al., 18: 153-4 (2000); Kurtzberg et al., Exp Hematol 26(4):288-98 (April 1998). The disclosures of the cited references are hereby incorporated by reference in their entireties.

One having ordinary skill in the art would appreciate that the above examples of media are illustrative and in no way limit the formulation of media suitable for use in the present invention and that there are many suitable media known and available to those in the art.

In various embodiments, the pharmaceutical composition of the invention includes a sterile solution of human serum albumin (HSA), such as 5% HSA, and low molecular weight (LMW) dextran.

The pharmaceutical composition is substantially free of mycoplasm, endotoxin, and microbial contamination. In particular embodiments, the therapeutic composition contains less than about 10, 5, 4, 3, 2, 1, 0.1, or 0.05 μg/ml bovine serum albumin.

With respect to mycoplasma and microbial contamination, “substantially free” as used herein means a negative reading for the generally accepted tests known to those skilled in the art. For example, mycoplasma contamination is determined by subculturing a sample of the therapeutic composition in broth medium and distributed over agar plates on day 1, 3, 7, and 14 at 37° C. with appropriate positive and negative controls. The sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The sample is considered satisfactory if the agar and/or the broth media procedure and the indicator cell culture procedure show no evidence of mycoplasma contamination.

An organic solvent or a suitable organic solvent relates generally to carbon containing liquids or gases that dissolve a solid, liquid, or gaseous solute, resulting in a solution. A suitable organic solvent is one that is appropriate for ex vivo administration to, or incubation with, mammalian cells, and can also be appropriate for in vivo administration to a subject, such as by having minimal toxicity or other inhibitory effects under ex vivo conditions (e.g., cell culture) or in vivo at a selected concentration for the time of incubation or administration. A suitable organic solvent should also be appropriate for storage stability and handling of the agents described herein.

Examples of suitable organic solvents include, but are not limited to, dimethyl sulfoxide (DMSO), N,N-dimethylformamide (DMF), dimethoxyethane (DME), and dimethylacetamide, including mixtures or combinations thereof. In certain embodiments, a composition or organic solvent is substantially free of methyl acetate, meaning that there should be no more than trace amounts of methyl acetate in the composition or solvent, and preferably undetectable amounts (e.g., as measured by high pressure liquid chromatography (HPLC), gas chromatography (GC), etc.).

A vessel or composition that is endotoxin free means that the vessel or composition contains at most trace amounts (i.e., amounts having no adverse physiological effects to a subject) of endotoxin, or undetectable amounts of endotoxin. Cells being “substantially free of endotoxin” means that there is less endotoxin per dose of cells than is allowed by the FDA for a biologic, which is a total endotoxin of 5 EU/kg body weight per day, which for an average 70 kg person is 350 EU per total dose of cells.

In one embodiment, the endotoxin free vessel and/or compositions is at least 95%, at least 96%, at least 97%, at least 98%, at least 99%, or 100% endotoxin free. Endotoxins are toxins associated with certain bacteria, typically gram-negative bacteria, although endotoxins can be found in gram-positive bacteria, such as Listeria monocytogenes. The most prevalent endotoxins are lipopolysaccharides (LPS) or lipooligosaccharides (LOS) found in the outer membrane of various Gram-negative bacteria, and which represent a central pathogenic feature in the ability of these bacteria to cause disease. Small amounts of endotoxin in humans can produce fever, a lowering of the blood pressure, and activation of inflammation and coagulation, among other adverse physiological effects. Therefore, it is often desirable to remove most or all traces of endotoxin from drug product containers, because even small amounts can cause adverse effects in humans. Endotoxins can be removed from vessels using methods known in the art, for example, vessels can be cleaned in HEPA filtered washing equipment with endotoxin-free water, depyrogenated at 250° C., and clean-packaged in HEPA filtered workstations located inside a class 100/10 clean room (e.g., a class 100 clean room, contains no more than 100 particles bigger than half a micron in a cubic foot of air).

In some embodiments, the pharmaceutical composition is administration ready, or ready for infusion, which means that the composition does not require any further treatment or manipulations prior to transplant or administration to a subject.

The present invention provides methods of ex vivo modulating an isolated population or subpopulation of T lymphocyte under conditions sufficient to improve its therapeutic properties. The methods provided in this invention can improve the isolated population or subpopulation of T lymphocytes for cell based therapies. In some embodiments, the ex vivo modulation decreases the potential of the isolated population or subpopulation of T lymphocytes to produce inflammatory cytokine or initiate an allogeneic response in a transplant recipient, therefore decreasing the risk of the recipient to develop GvHD. In other embodiments, the invention provides compositions comprising a population or subpopulation of modulated T lymphocytes having increased persistence in a transplant subject relative to an unmodulated population or subpopulation of T lymphocytes.

In some embodiments, the present invention provides a method of modulating an isolated population or subpopulation of T lymphocytes by contacting the isolated population of T lymphocytes ex vivo with an agent selected from a prostaglandin pathway agonist, a glucocorticoid, or a combination thereof, wherein the isolated population of T lymphocytes comprises peripheral blood mononuclear cells (PBMC), peripheral blood leukocytes (PBL), tumor infiltrating lymphocytes (TIL), or a combination thereof.

In some embodiments, the present invention provides a method of modulating an isolated population or subpopulation of T lymphocytes by contacting the isolated population of T lymphocytes ex vivo with an agent selected from a prostaglandin pathway agonist, a glucocorticoid, or a combination thereof, wherein the isolated population of T lymphocytes comprise cells selected from the group consisting of CD4+/CD8+ double positive T cells, cytotoxic T cells, Th3 (Treg) cells, Th9 cells, Thαβ helper cells, Tfh cells, stem memory T_{SCM} cells, central memory T_{CM} cells, effector memory T_{EM} cells, effector memory T_{EMRA} cells, gamma delta T cells and any combination thereof.

In some embodiments, the present invention provides a method of modulating an isolated population or subpopulation of T lymphocytes by contacting the isolated population or subpopulation of T lymphocytes ex vivo with an agent selected from a prostaglandin pathway agonist, a glucocorticoid, or a combination thereof, wherein the isolated population or subpopulation of T lymphocytes comprise two or more types of T lymphocytes selected from the group consisting of CD4+/CD8+ double positive T cells, CD4+ T cells, CD8+ T cells, naive T cells, effector T cells, cytotoxic T cells, helper T cells, memory T cells, regulator T cells, Th0 cells, Th1 cells, Th2 cells, Th3 (Treg) cells, Th9 cells, Thαβ helper cells, Tfh cells, stem memory T_{SCM} cells, central memory TCM cells, effector memory T_{EM} cells, effector memory T_{EMRA} cells, gamma delta T cells and any combination thereof.

In some embodiments, the present invention provides a method of modulating an isolated population or subpopulation of T lymphocytes by contacting the isolated population or subpopulation of T lymphocytes ex vivo with an agent having a prostaglandin pathway agonist, a glucocorticoid, or a combination thereof, wherein the isolated population of T lymphocytes is differentiated in vitro from an stem cell or progenitor cell.

In some embodiments, the present invention provides a method of modulating an isolated population or subpopulation of T lymphocytes by contacting the isolated population or subpopulation of T lymphocytes ex vivo with an agent selected from a prostaglandin pathway agonist, a glucocorticoid, or a combination thereof, wherein the isolated population or subpopulation of T lymphocytes have an exogenous nucleic acid. In some aspects, the exogenous nucleic acid can encode a TCR or a CAR.

In some embodiments, the present invention provides a method of modulating an isolated population or subpopulation of T lymphocytes comprising contacting the isolated population or subpopulation of T lymphocytes ex vivo with a prostaglandin pathway agonist and a glucocorticoid.

In some embodiments, the methods also include washing the modulated population or subpopulation of T lymphocytes to remove the treatment agents. In some aspects, the modulated population or subpopulation of T lymphocytes is washed with a buffer that is substantially free of the agent.

The method of the invention can be applied to T lymphocyte of any sources as described herein. For example, the isolated population or subpopulation of T lymphocytes can be isolated from a living organism, such as a human. The isolated population or subpopulation of T lymphocyte can be obtained from peripheral blood, cord blood or bone marrow.

In some embodiments, the isolated population or subpopulation of T lymphocytes can also be differentiated from stem cells or progenitor cells, such as an iPSC or ESC. In some aspects, the pluripotent stem cell is differentiated into T lymphocytes with at least one of a BMP pathway activator, a WNT pathway activator, and an extracellular matrix protein. In some aspects, the pluripotent stem cell can be maintained under feeder free conditions.

In some embodiments, the pluripotent stem cell is differentiated into a mesoderm cell that further differentiates into the isolated population or subpopulation of T lymphocytes. In some aspects, the pluripotent stem cell is differentiated into a definitive hematopoietic stem cell that further differentiates into the isolated population or subpopulation of T lymphocytes. In some aspects, the pluripotent stem cell is differentiated into a T lymphocyte progenitor that further differentiates into the isolated population or subpopulation of T lymphocytes. In some aspects, the T lymphocyte progenitor cell is maintained under feeder free conditions for maturation to the isolated population or subpopulation of T lymphocytes.

The isolated population or subpopulation of T lymphocytes can also be genetically modified to have an exogenous polynucleotide, such as an isolated population or subpopulation of T lymphocytes expressing a recombinant TCR or CAR. Accordingly, the present invention provides methods to modify any types of isolated, differentiated, or recombinant T lymphocytes as described herein.

The present invention provides a method of modulating an isolated population or subpopulation of T lymphocytes by contacting said T lymphocytes ex vivo with an agent that is a prostaglandin pathway agonist, a glucocorticoid, or a combination thereof. Described below are exemplary embodiments of the methods of the present invention. A person of ordinary skill in the art would understand that the methods of the present invention including treating any and all types of isolated population or subpopulation of T lymphocytes as described herein, under any conditions as described herein to improve the therapeutic of the isolated population or subpopulation of T lymphocytes. The conditions include, but are not limited to, the agent used and the concentrations thereof, as described above, the time the cells are exposed to the agent, and the temperature of treatment, as described herein.

In some embodiments, the agent used to modulate the isolated population or subpopulation of T lymphocytes ex vivo is a combination of a prostaglandin pathway agonist and a glucocorticoid. In some embodiments, the prostaglandin pathway agonist is a PGE receptor agonist. In some embodiments, the PGE receptor agonist can be a compound that selectively binds the PGE₂ EP₂ or PGE₂ EP₄ receptor. In some embodiments, the PGE receptor agonist can be a PGE₂, or a PGE₂ analogue or derivative. In some embodiments, the PGE receptor agonist can be a prostaglandin pathway agonist selected from the group consisting of PGE₂, dmPGE₂, 15(S)-15-methyl PGE₂, 20-ethyl PGE₂, and 8-iso-16-cyclohexyl-tetranor PGE₂.

In some embodiments, the prostaglandin pathway agonist is selected from the group consisting of is PGE₂, prostaglandin I₂, 16,16-dimethyl PGE₂ (dmPGE₂); 16,16-dimethyl PGE₂ p-(p-acetamidobenzamido) phenyl ester; 11-deoxy-16,16-dimethyl PGE₂; 9-deoxy-9-methylene-16,16-dimethyl PGE₂; 9-deoxy-9-methylene PGE₂; 9-keto Fluprostenol; 5-trans PGE₂; 17-phenyl-omega-trinor PGE₂; PGE₂ serinol amide; PGE₂ methyl ester; 16-phenyl tetranor PGE₂; 15(S)-15-methyl PGE₂; 15(R)-15-methyl PGE₂; 8-iso-15-keto PGE₂; 8-iso PGE₂ isopropyl ester; 8-iso-16-cyclohexyltetranor PGE₂; 20-hydroxy PGE₂; 20-ethyl PGE₂; 11-deoxy PGE₁; nocloprost; sulprostone; butaprost; 15-keto PGE₂; 19(R) hydroxy PGE₂; 5-[(1E,3R)-4,4-difluoro-3-hydroxy-4-phenyl-1-buten-1-yl]-1-[6-(2H-tetrazol-5R-yl)hexyl]-2-pyrrolidinone (L-902,688); 2-[3-[(1R,2S,3R)-3-hydroxy-2-[(E,3S)-3-hydroxy-5-[2-(methoxymethyl)phenyl]pent-1-enyl]-5-oxocyclopentyl]sulfanylpropylsulfanyl]acetic acid (ONO-AE1-329); methyl4-[2-[(1R,2R,3R)-3-hydroxy-2-[(E,3S)-3-hydroxy-4-[3-(methoxymethyl)phenyl]but-1-enyl]-5-oxocyclopentyl]ethylsulfanyl]butanoate (ONO-4819); 16-(3-Methoxymethyl)phenyl-co-tetranor-5-thiaPGE₁; 5-{3-[(2S)-2-{(3R)-3-hydroxy-4-[3-(trifluoromethyl)phenyl]butyl}-5-oxopyrrolidin-1-yl]propyl]thiophene-2-carboxylate (PF-04475270); APS-999 Na; [4′-[3-butyl-5-oxo-1-(2-trifluoromethyl-phenyl)-1,5-dihydro-[1,2,4]triazol-4-ylmethyl]-biphenyl-2-sulfonic acid (3-methyl-thiophene-2-carbonyl)-amide]; ((Z)-7-{(1R,4S,5R)-5-[(E)-5-(3-chloro-benzo[b]thiophene-2-yl)-3-hydroxy-pent-1-enyl]-4-hydroxy-3,3-dimethyl-2-oxo-cyclopentyl})-hept-5-enoic acid); Linoleic Acid; 13(s)-HODE; LY171883; Mead Acid; Eicosatrienoic Acid; Epoxyeicosatrienoic Acid; ONO-259; Cay1039; Butaprost; Sulprostone; CAY10399; ONO_8815Ly; ONO-AE1-259; or CP-533,536. In some embodiments, the prostaglandin pathway agonist is dmPGE₂.

In some embodiments, the glucocorticoid is selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, or ulobetasol. In some embodiments, the glucocorticoid is dexamethasone.

In some embodiments, the present invention provides a method to ex vivo modulate a population or subpopulation of T lymphocytes with dmPGE₂ and dexamethasone. In some embodiments, the concentration of the prostaglandin pathway agonist is about 10 μm. In some embodiments, the concentration of the glucocorticoid is about 10 μm.

In some embodiments, the present invention provides a method to contact isolated population or subpopulation of T lymphocytes with ex vivo an agent to improve its therapeutic potential for a period of time that is at least about one hour. In some embodiments, the period of time is at least about one hour to about 24 hours. In some embodiments, the period of time is at least about one hour to about twelve hours. In some embodiments, the period of time is at least about one hour to about six hours. In some embodiments, the period of time is at least about one hour to about four hours. In some embodiments, the period of time is at least about two hours to about four hours.

In some embodiments, the method of present invention includes contacting an isolated population or subpopulation of T lymphocytes ex vivo with an agent to improve its therapeutic potential at a temperature that is between about 24° C. to about 39° C. In some embodiments, the method includes contacting the isolated population or subpopulation of T lymphocytes ex vivo with an agent at about 37° C. The methods of the present invention are not limited to these exemplary embodiments with specific conditions. Rather, the present invention includes treating the isolated population or subpopulation of T lymphocytes under any conditions as described herein in connection with the isolated population or subpopulation of T lymphocytes and compositions of the invention.

In some embodiments, the present invention provides a method of modulating an isolated population or subpopulation of T lymphocyte by contacting the isolated population or subpopulation of T lymphocytes ex vivo with an agent having a prostaglandin pathway agonist, a glucocorticoid, or a combination thereof, wherein the expression of a signature gene in the isolated population or subpopulation of T lymphocytes is increased by at least about two fold compared to a noncontacted population or subpopulation of T lymphocytes. In some embodiments, the signature gene can be selected from the group consisting of BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, IL21R, MCAM, SOCS1, TNFSF4, TOB1, TSC22D3, FGL2, NR4A2, AREG, TGFB1, CD55, THFAIP3, and CXCR4. In some embodiments, the signature gene can be selected from the group consisting of FGL2, NR4A2, AREG, TGFB1, CD55, CCR4, THFAIP3, NLRP3, CTLA4, and CXCR4. In some other embodiments, the signature gene can be selected from the group consisting of Plaur, Fosl2, Ccr8, Areg, Nr4a2, Pdcd1, Nr4a3, and Ctla4. In some embodiments, the signature gene can be selected from the group consisting of ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2.

In some embodiments, the expression of at least two signature genes are increased by at least two fold. In some embodiments, the expression of at least three, five, ten, or fifteen signature genes are increased by at least two fold. In some embodiments, the expression of one or more signature genes is increased by at least 3, 5, or 10 fold compared to a noncontacted population or subpopulation of T lymphocytes.

In some embodiments, the present invention provides a method of modulating an isolated population or subpopulation of T lymphocytes comprising contacting the isolated population or subpopulation of T lymphocytes ex vivo with an agent selected from a prostaglandin pathway agonist, a glucocorticoid, or a combination thereof, wherein the isolated population or subpopulation of T lymphocytes have increased level of PD-1 at cell surface compared to a noncontacted population of T lymphocyte. In some embodiments, the isolated population of T lymphocyte has a decreased level of ICOS or 41BB at cell surface compared to a noncontacted population or subpopulation of T lymphocytes. In some embodiments, the isolated population or subpopulation of T lymphocytes have decreased production of interferon gamma compared to a noncontacted population or subpopulation of T lymphocytes, or more production of interleukin 4 or interleukin 10 compared to a noncontacted population or subpopulation of T lymphocytes. In some embodiments, the present invention provides a method of modulating an isolated population or subpopulation of T lymphocytes by contacting the isolated population or subpopulation of T lymphocytes ex vivo with an agent selected from a prostaglandin pathway agonist, a glucocorticoid, or a combination thereof, wherein the isolated population or subpopulation of T lymphocytes have reduced proliferation capacity, or reduced allogeneic response compared to a noncontacted population or subpopulation of T lymphocytes. Accordingly, the present invention provides methods of ex vivo modulation of an isolated population or subpopulation of T lymphocytes that have resulted in a change in expression of signature genes or other properties such as production of certain cytokines or any permutation or combination of the effects as described herein.

The present invention also provides methods of treating a patient in need thereof by administrating the modulated population or subpopulation of T lymphocytes of the invention, or the therapeutic composition having the same to a subject in need thereof. The modulated population or subpopulation of T lymphocytes, pharmaceutical compositions and methods of the present invention can be used to treat a subject in need of cell therapies. The subject can be a human. The subject can be a candidate for bone marrow or stem cell transplantation, or has received chemotherapy or irradiation therapy. In some embodiments, the subject has received bone marrow ablative or non-myeolablative chemotherapy or radiation therapy. The cell therapy can be adoptive cell therapy.

The subject can also be a cancer patient, or patient with a hyperproliferative disorder. In some embodiments, the subject has a hyperproliferative disorder of the hematopoietic system. In some embodiments, the subject has a solid tumor. In some aspects, the hyperproliferative disorder of the hematopoietic system is polycythemia vera, essential thrombocythemia, myelofibrosis with myeloid metaplasia, or chronic myelogenous leukemia.

The isolated population or subpopulation of T lymphocytes or pharmaceutical compositions of the present invention can be used to treat cancers. Cancers that can be treated include tumors that are not vascularized, or not yet substantially vascularized, as well as vascularized tumors. The cancers can be non-solid tumors (such as hematological tumors, for example, leukemias and lymphomas) or can be solid tumors. Types of cancers to be treated include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

In some embodiments, the modulated population or subpopulation of T lymphocytes or pharmaceutical compositions of the present invention are used to treat hematologic cancers. Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia, chronic myeloic leukemia, and chronic lymphocytic leukemia), juvenile myelomonocytic leukemia, polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, agnogenic myeloid metaplasia, familial erythrophagocytic lymphohistiocytosis, hairy cell leukemia and myelodysplasia.

In some embodiments, the subject has myeloma, non-Hodgkin's lymphoma, Hodgkin's lymphoma, chronic myeloid leukemia, chronic myelogenous leukemia, chronic granulocytic leukemia, acute lymphoblastic leukemia, acute nonlymphoblastic leukemia, or pre-leukemia.

In some embodiments, the modulated population or subpopulation of T lymphocytes or pharmaceutical compositions of the present invention are used to treat solid tumors. Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, meduloblastoma, Schwannoma craniopharyogioma, ependymoma, pineaioma, hemangioblastoma, acoustic neuroma, oligodendroglioma, menangioma, neuroblastoma, retinoblastoma and brain metastases).

In some embodiments, the subject has breast cancer, ovarian cancer, brain cancer, prostate cancer, lung cancer, colon cancer, skin cancer, liver cancer, pancreatic cancer, sarcoma, or chronic granulomatous disease.

The subject can also be a patient having viral infection, or a patient having a disease associated with viral infection. The disease associated with virus infection can be HIV/AIDS, chicken pox, cervical cancer, hepatitis (e.g., hepatitis A, hepatitis B, hepatitis C), Hodgkin's lymphoma, influenza, measles, mumps, poliomyelitis, rabies, viral hemorrhagic fever, or viral meningitis. The viral infections further include, but are not limited to, HIV—(human immunodeficiency virus), RSV—(Respiratory Syncytial Virus), EBV—(Epstein-Barr virus), CMV—(cytomegalovirus), adenovirus- and BK polyomavirus-associated disorders.

Other subjects can have disorders caused by an infection (e.g., viral infection, bacterial infection or fungal infection) which causes damage to the bone marrow.

In addition, a subject suffering from the following conditions can also benefit from treatment using the T lymphocytes of the invention: lymphocytopenia, lymphorrhea, lymphostasis, erythrocytopenia, erthrodegenerative disorders, erythroblastopenia, leukoerythroblastosis; erythroclasis, thalassemia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated-neutropenias, childhood and adult cyclic neutropaenia; post-infective neutropaenia; myelodysplasia syndrome; neutropaenia associated with chemotherapy and radiotherapy; chronic granulomatous disease; mucopolysaccharidoses; Diamond Blackfan; Sickle cell disease; or Beta thalassemia major.

A person of ordinary skill in the part would understand that the present invention provides methods to treat the indications as described herein with modulated, isolated population or subpopulation of T lymphocytes of instant invention, wherein the isolated population or subpopulation of T lymphocytes can be obtained from a variety of sources as described herein. In some embodiments, the isolated population or subpopulation of T lymphocytes are obtained from a living organism, such as human cord blood or peripheral blood. In some embodiments, the isolated population or subpopulation of T lymphocytes are differentiated from an iPSC or ESC. In some embodiments, the isolated population or subpopulation of T lymphocytes are genetically engineered to produce a recombinant TCR or CAR to specifically recognize tumor antigens.

In some embodiments, antigen bind moiety portion of the CAR of the modulated population or subpopulation of T lymphocytes of the invention is designed to treat a particular cancer. For example, the CAR designed to target CD19 can be used to treat cancers and disorders including but are not limited to pre-B ALL (pediatric indication), adult ALL, mantle cell lymphoma, diffuse large B-cell lymphoma, salvage post allogenic bone marrow transplantation, and the like. In another embodiment, the CAR can be designed to target CD22 to treat diffuse large B-cell lymphoma. In one embodiment, cancers and disorders include but are not limited to pre-B ALL (pediatric indication), adult ALL, mantle cell lymphoma, diffuse large B-cell lymphoma, salvage post allogenic bone marrow transplantation, and the like can be treated using a combination of CARs that target CD19, CD20, CD22, and ROR1. In one embodiment, the CAR can be designed to target mesothelin to treat mesothelioma, pancreatic cancer, ovarian cancer, and the like. In one embodiment, the CAR can be designed to target CD33/IL3Ra to treat acute myelogenous leukemia and the like. In one embodiment, the CAR can be designed to target c-Met to treat triple negative breast cancer, non-small cell cancer, and the like. In one embodiment, the CAR can be designed to target PSMA to treat prostate cancer and the like. In one embodiment, the CAR can be designed to target Glycolipid F77 to treat prostate cancer and the like. In one embodiment, the CAR can be designed to target EGFRvIII to treat gliobastoma and the like. In one embodiment, the CAR can be designed to target GD-2 to treat neuroblastoma, melanoma, and the like. In one embodiment, the CAR can be designed to target NY-ESO-1 TCR to treat myeloma, sarcoma, melanoma, and the like. In one embodiment, the CAR can be designed to target MAGE A3 TCR to treat myeloma, sarcoma, melanoma, and the like.

The modulated population or subpopulation of T lymphocytes of the invention is not limited to those expressing the CARs specific to the antigen targets and diseases disclose herein. Rather, the modulated population or subpopulation of T lymphocytes of the invention is understood to include, but not be limited to, any isolated population or subpopulation of T lymphocytes, including those that are genetically engineered to target a specific disease, wherein the modulated population or subpopulation of T lymphocytes have been contacted with an agent that is a prostaglandin pathway agonist, a glucocorticoid, or a combination thereof, under conditions sufficient to improve the therapeutic properties thereof.

In various embodiments, the present invention provides, in part, methods including administration of a modulated population or subpopulation of T lymphocytes, or a pharmaceutical composition thereof, to a subject in need thereof. Suitable methods for administering populations of T lymphocytes used in the methods described herein include parenteral administration, including, but not limited to methods of intravascular administration, such as intravenous and intraarterial administration. Additional illustrative methods for administering cells of the invention include intramuscular, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

In some embodiments, the modulated population or subpopulation of T lymphocytes, or the pharmaceutical composition thereof, are administered or infused to a subject intravenously.

The modulated population or subpopulation of T lymphocytes or the pharmaceutical composition of the invention can be inserted into a delivery device which facilitates introduction by injection or implantation into the subjects. Such delivery devices include, but are not limited to, tubes, e.g., catheters, for injecting cells and fluids into the body of a recipient subject. In one embodiment, the tubes additionally have a needle, e.g., a syringe, through which the T lymphocytes of the invention can be introduced into the subject at a desired location. In a particular embodiment, cells are formulated for administration into a blood vessel via a catheter (where the term “catheter” is intended to include any of the various tube-like systems for delivery of substances to a blood vessel).

It is understood that modifications which do not substantially affect the activity of the various embodiments of this invention are also provided within the definition of the invention provided herein. Accordingly, the following examples are intended to illustrate but not limit the present invention.

### Example I

### Gene Expression Panel of CD3+ T-cell Treated with dmPGE₂

RNA was extracted from CD3+ T-cells pre-isolated from human peripheral blood (AllCells, Aiameda CA) (n=3 donors) post ex vivo treatment (2 hours at 37° C. in StemSpan-ACF (StemCell Technologies, Vancouver, Canada)) with 10 μM dmPGE₂ (Fate therapeutics, San Diego, Calif.) or vehicle control (DMSO).

Following cell treatments, levels mRNA were quantitated from PicoPure isolated mRNA (Life Technologies, Carlsbad, Calif.) using Affymetrix GeneChip technology. Affymetrix U133 plus 2.0 GeneChip hybridization of all samples was performed in accordance with the manufacturer's recommendations (Affymetrix, Santa Clara, Calif.), involving roughly 200 ng of total RNA and Message Amp II (Life Technologies, Carlsbad, Calif.) standard two round amplification protocols. Probe intensities were normalized according to a log scale robust multi-array analysis (RMA) method (Affymetrix Santa Clara, Calif.). A parametric paired t-test (Benjamini-Hochberg false discovery rate <0.05, adjusted p-value/q-value <0.05, and fold change > or <2 fold) detected probes with significant changes due to the treatment with dmPGE2 and dexamethasone.

The probe intensities were converted into linear fold changes of relative expression levels of mRNA by comparison of dmPGE2 and dexamethasone treated CD3+ T-cells in comparison to the vehicle treated control samples. Results are the mean of three individual donors of CD3+ T cells. Plotted are the average linear fold changes in expression from a panel of key biologically relevant genes across the 3 donor samples (Microsoft Excel).

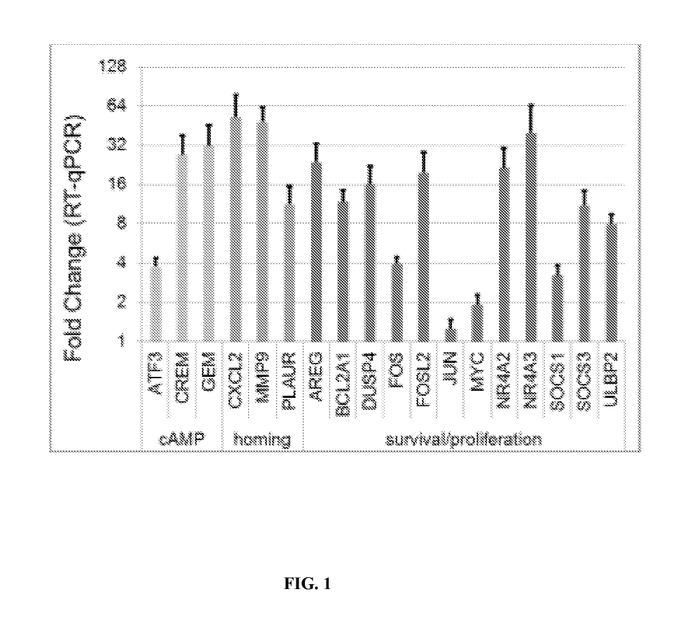

As shown FIG. 1, at least the following genes have demonstrated a statistically significant increase (at least 2 fold) in expression when the isolated CD3+ T-cells are treated with dmPGE2, including ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2.

### Example II

### Gene Expression Panel of CD3+ T-cells Treated with Compound Combination

RNA was extracted from CD3+ T-cells pre-isolated from human peripheral blood (AllCells) (n=3 donors) post ex vivo treatment (4 hours at 37° C. in StemSpan-ACF (StemCell Technologies)) with 10 M dmPGE₂ (Fate therapeutics) and 10 M Dexamethasone (Sigma, St. Louis, Mo.) or vehicle control (DMSO).

Following cell treatments, levels mRNA were quantitated from PicoPure isolated mRNA (Life Technologies) using Affymetrix GeneChip technology. Affymetrix U133 plus 2.0 GeneChip hybridization of all samples was performed in accordance with the manufacturer's recommendations (Affymetrix), involving roughly 200 ng of total RNA and Message Amp II (Life Technologies) standard two round amplification protocols. Probe intensities were normalized according to a log scale robust multi-array analysis (RMA) method (Affymetrix). A parametric paired t-test (Benjamini-Hochberg false discovery rate <0.05, adjusted p-value/q-value <0.05, and fold change > or <2 fold) detected probes with significant changes due to the treatment with dmPGE2 and dexamethasone.

The probe intensities were converted into linear fold changes of relative expression levels of mRNA by comparison of dmPGE2 and dexamethasone treated CD3+ T-cells in comparison to the vehicle treated control samples. Results are the mean of three individual donors of CD3+ T cells. Plotted are the average linear fold changes in expression from a panel of key biologically relevant genes across the 3 donor samples (Microsoft Excel).

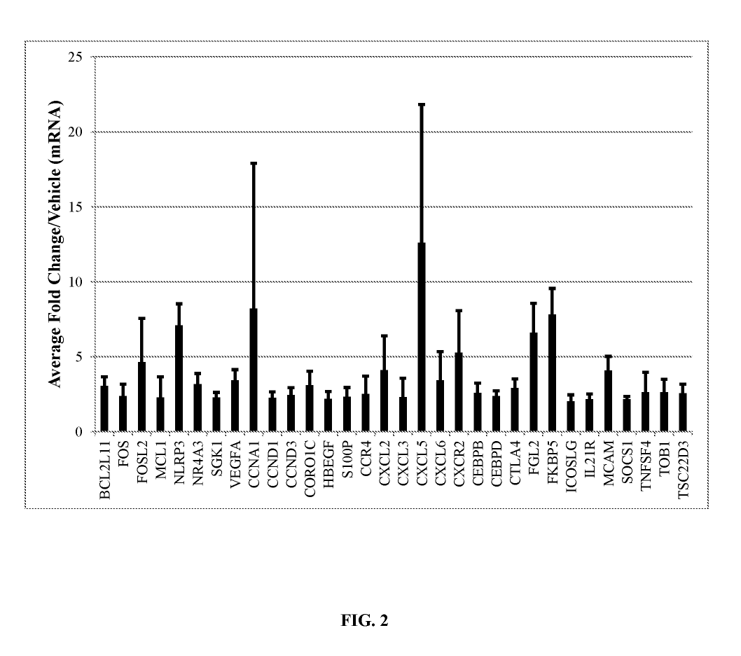

As shown FIG. 2, at least the following genes have demonstrated a statistically significant increase (at least 2 fold) in expression when the isolated CD3+ T-cells are treated with a combination of dmPGE2 and dexamethasone, including BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, IL21R, MCAM, SOCS1, TNFSF4, TOB1, and TSC22D3.

### Example III

### Effects of dmPGE2 and Dexamethasone on T Lymphocytes

The example illustrates the effects of combination dmPGE2 and dexamethasone on T cells in a mobilized peripheral blood product.

Donor mobilized peripheral blood leukopaks were obtained from a number of commercial suppliers (a total of 7 donors obtained over a 3 month period) and modulated with 10 M dmPGE2 and 10 M dexamethasone for 4 hours at 37° C. After the 4 hour modulation, the cells were washed extensively to remove the compounds and then plated for a number of in vitro T cell assays to assess function. All T cell assays included 5 day incubations with readouts on day 6 after compound washout. Thus, the results demonstrate that pulse treatment of modulators can have lasting effects on T cells.

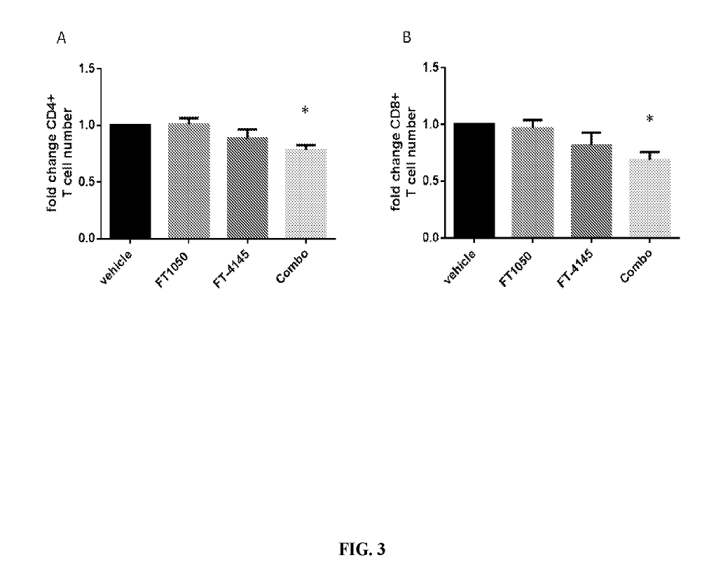

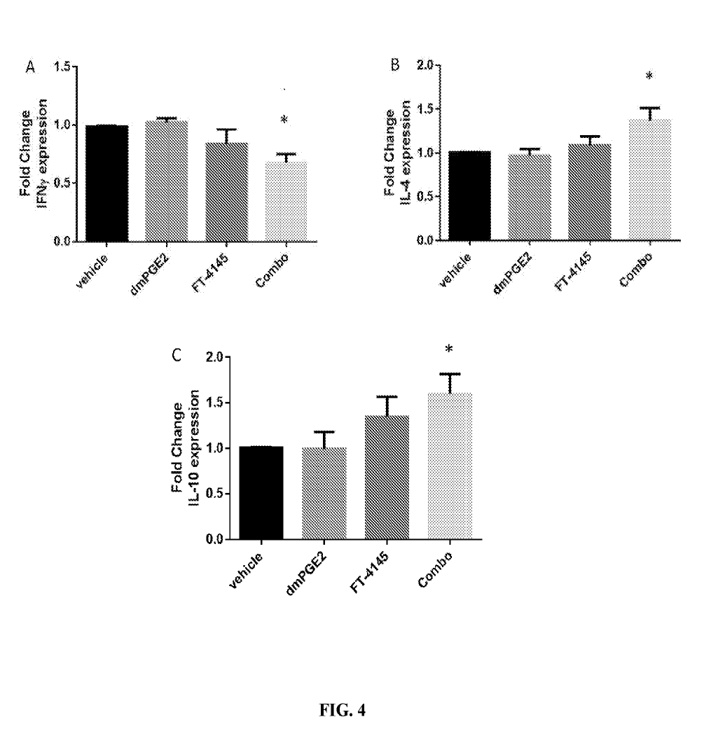

As shown in FIG. 3, when T cells were co-incubated with mismatched peripheral blood mononuclear cells from an alternate donor, these cells (both CD4 and CD8) were significantly less capable of proliferating and therefore had a reduced allogeneic response. Additionally, these cells were significantly less proficient at producing a key effector cytokine, Interferon gamma, but more proficient at producing both interleukin 4, a less inflammatory cytokine, and interleukin 10 (a well-established anti-inflammatory cytokine) (FIGS. 4a-c).

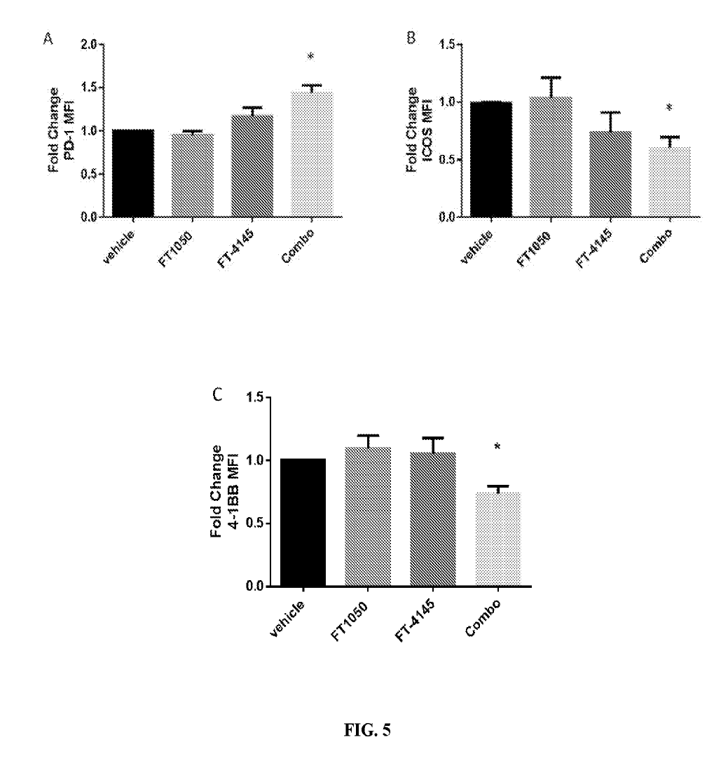

The modulated T cells expressed higher levels of PD-1, and lower levels of ICOS and 41BB protein at their cell surface (FIGS. 5a-c). The expression levels of these particular proteins suggests that these T cells are significantly less capable of becoming activated in response to T cell receptor engagement.

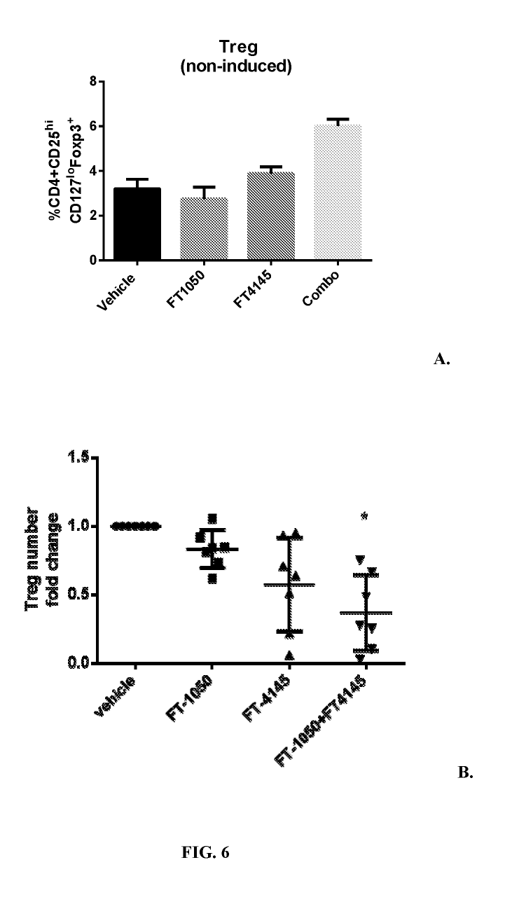

Expansion of T regulatory cells was enhanced following modulation and activation (FIG. 6A) using the compound combination; however, was reduced in response to polarization conditions containing transforming growth factor B when modulated (FIG. 6B). Taken together, these data suggest that modulation of a mobilized peripheral blood product with dmPGE2 and dexamethasone has an effect of programming the effector T cell compartment to reduce their activation state, which subsequently respond in a less robust manner to allogeneic targets.

### Example IV

### Genome-wide Expression Panel of Tregs

Pre-isolated cryopreserved CD4+CD25+ T-cells from human peripheral blood (AllCells) or sorted mouse cells from spleens of Foxp3-GFP B6 Mice using a FACSARIA (Becton Dickenson, Franklin Lakes, N.J.) were ex vivo treated (4 hours at 37° C. in StemSpan-ACF (StemCell Technologies)) with 10 M dmPGE2 (Fate therapeutics) and 10 M Dexamethasone (Sigma) or vehicle control (DMSO). T cells treated with dmPGE2 and Dexamethasone have an increased polarization toward Tregs.

RNA was extracted using the PicoPure RNA Isolation kit (Life Technologies) using the manufacturers recommended protocol. Total RNA was quantified using the Nanodrop 2000 Spectrophotometer (Thermo Scientific). RNA integrity was confirmed using an Agilent 2100 Bioanalyzer (Agilent Technologies, La Jolla, Calif.).

Affymetrix HTA 2.0 (human) and MTA 1.0 (mouse) GeneChip hybridization of all samples were performed in accordance with the manufacturer's recommendations (Affymetrix, Santa Clara, Calif.), involving roughly 1-3 ng of total RNA input, using a single high concentration cRNA amplification round followed by cDNA array hybridization. Probe intensities were normalized according to a log scale robust multi-array analysis (RMA) method (Expression Console, Affymetrix) and analyzed for differential expression (Transcriptome Analysis Console v3.0, Affymetrix). Scatterplot visualizations of normalized probe intensities comparing treated versus control cells were created with TIBCO Spotfire 6.0 (Perkin-Elmer, Waltham, Mass.).

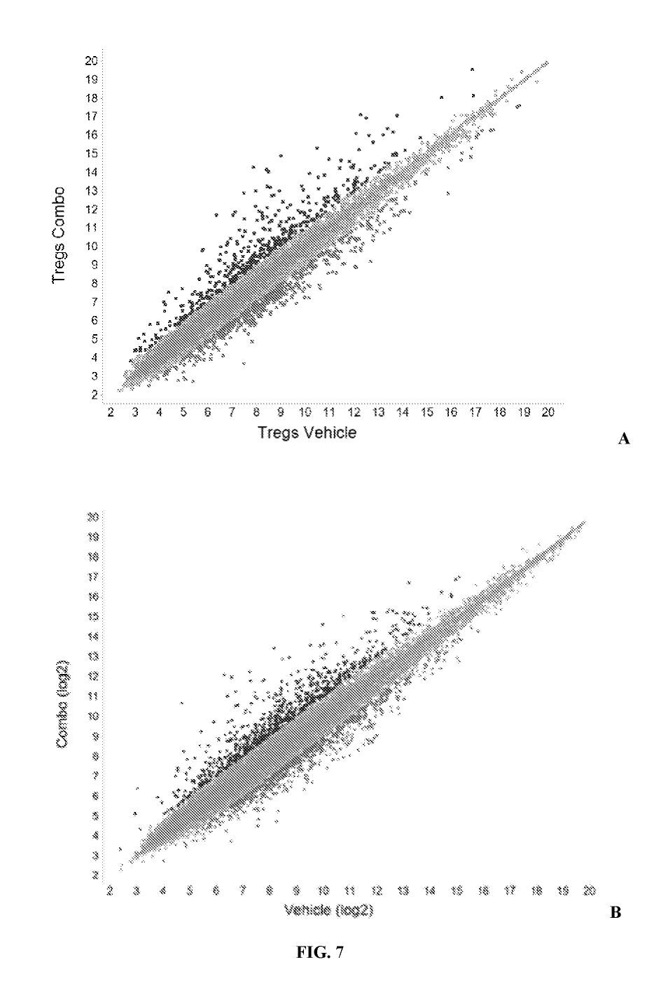

To determine the impact of treatment with dmPGE2 and dexamethasone on regulatory T cells, a genome-wide microarray approach was used to analyze total RNA isolated from both human and mouse cells by comparing the treated samples to corresponding untreated control samples. The average RMA intensity (log 2) of probes hybridized from the dmPGE2 and dexamethasone treated cells are plotted on the Y-axis, while the control (vehicle only) treated samples are on the X-axis (FIG. 7, A-human B-mouse). Probes (markers specific to individual gene transcripts) that have an increase in transcriptional expression due to treatment with dmPGE2 and dexamethasone are highlighted in black while grey highlighted probes have reduced expression due to the compound treatment. The normalized probe intensities were converted into linear fold changes of relative expression levels of mRNA by comparison of dmPGE2 and dexamethasone treated regulatory T cells in comparison to the vehicle treated control samples for both human and mouse. Table 1 indicates the average linear fold changes in expression from a panel of a few key biologically relevant genes (Table 1A-human; 1B-mouse). These and other observed increases in gene expression levels are believed to indicate an enhanced regulatory T cell function and improved therapeutic potential. Among the enhanced Treg effector moleculs, for example, Plaur is a critical gene for Treg cell suppressive function (also known as suppressor function) so that the proliferation of other activated T cells, including CD4+ and CD8+, and the production of cytokine and cytotoxicity thereof are limited, Ccr8 potentiates donor Treg survival and has been demonstrated to play a role in the attenuation of graft-versus host disease; and FGL2 works as a novel effector of Treg, demonstrating immunoregulatory function to protect against tissue injuries.

TABLE 1
Expression fold changes of exemplary key biologically relevant genes

| | | | |
|---|---|---|---|
| Fold | Gene | UniProtKB | |
| Change | Symbol | Entry No. | Description |

A: Human

| | | | |
|---|---|---|---|
| 87.14 | FGL2 | Q14314 | fibrinogen-like 2 |
| 25.67 | NR4A2 | P43354 | nuclear receptor subfamily 4, |
| 16.67 | AREG | P15514 | amphiregulin; amphiregulin B |
| 7.83 | TGFB1 | P01137 | transforming growth factor, beta 1 |
| 7.27 | CD55 | P08174 | CD55, decay accelerating factor |
| 7 | CCR4 | P51679 | chemokine (C-C motif) receptor 4 |
| 6.54 | TNFAIP3 | P21580 | tumor necrosis factor,alpha-induced |
| | | | protein 3 |
| 3.7 | NLRP3 | Q96P20 | NLR family, pyrin domain containing |
| | | | 3 |
| 2.78 | CTLA4 | P16410 | cytotoxic T-lymphocyte-associated |
| | | | protein 4 |
| 2.4 | CXCR4 | P61073 | chemokine (C—X—C motif) receptor |
| | | | 4 |

B: Mouse

| | | | |
|---|---|---|---|
| 32.54 | Plaur | P35456 | plasminogen activator, urokinase |
| | | | receptor |
| 21.86 | Fosl2 | P47930 | fos-like antigen 2 |
| 19.4 | Ccr8 | P56484 | chemokine (C-C motif) receptor 8 |
| 14.25 | Areg | P31955 | amphiregulin |
| 13.09 | Nr4a2 | Q06219 | nuclear receptor subfamily 4, member |
| | | | 2 |
| 9.5 | Pdcd1 | Q9EP73 | programmed cell death 1 |
| 8.92 | Nr4a3 | Q9QZB6 | nuclear receptor subfamily 4, member |
| | | | 3 |
| 5.53 | Ctla4 | P09793 | cytotoxic T-lymphocyte-associated |
| | | | protein 4 |

We claim:

1. An isolated population of T lymphocytes that has been contacted ex vivo with a composition comprising at least one prostaglandin pathway agonist, or at least one glucocorticoid, or a combination thereof, wherein said contacted population of T lymphocytes comprises modulated cells
(i) exhibiting a decreased proliferation of CD4+ and/or CD8+ cells;
(ii) exhibiting decreased allogeneic response;
(iii) exhibiting increased persistence when transplanted; and/or
(iv) exhibiting altered cytokine production,
when compared to cells in a non-contacted isolated population of T lymphocytes.

2. The isolated population of T lymphocytes of claim 1, wherein said T lymphocytes
(i) comprise peripheral blood mononuclear cells (PBMC), peripheral blood leukocytes (PBL), tumor infiltrating lymphocytes (TIL), or a combination thereof, or
(ii) comprise one or more subpopulations selected from the group consisting of CD4+/CD8+ double positive T cells, CD4+ T cells, CD8+ T cells, CD3+ T cells, naive T cells, effector T cells, cytotoxic T cells, helper T cells, memory T cells, regulator T cells, Th0 cells, Th1 cells, Th2 cells, Th3 (Treg) cells, Th9 cells, Thαβ helper cells, Tfh cells, stem memory T_{SCM} cells, central memory T_{CM} cells, effector memory T_{EM} cells, effector memory T_{EMRA} cells, gamma delta T cells, and any combinations thereof; or
(iii) are derived from peripheral blood, cord blood, or bone marrow; or
(iv) are differentiated in vitro from a stem cell, a definitive hemogenic endothelium, a CD34+ cell, a hematopoietic stem and progenitor cell, a hematopoietic multipotent progenitor cell, or a T cell progenitor cell;
(v) comprise an exogenous nucleic acid; or
(vi) are human cells.

3. The isolated population of T lymphocytes of claim 2, wherein said stem cell is an induced pluripotent stem cell (iPSC) or an embryonic stem cell (ESC).

4. The isolated population of T lymphocytes of claim 2, wherein said exogenous polynucleotide encodes a TCR or a CAR.

5. The isolated population of T lymphocytes of claim 1, wherein said prostaglandin pathway agonist is
(i) a PGE receptor agonist;
(ii) PGE₂, or a PGE₂ derivative or analogue; or
(iii) a compound selected from the group consisting of PGE₂, prostaglandin 12, 16,16-dimethyl PGE₂ (dmPGE₂); 16,16-dimethyl PGE₂ p-(p-acetamidobenzamido) phenyl ester; 11-deoxy-16,16-dimethyl PGE₂; 9-deoxy-9-methylene-16,16-dimethyl PGE₂; 9-deoxy-9-methylene PGE₂; 9-keto Fluprostenol; 5-trans PGE₂; 17-phenyl-omega-trinor PGE₂; PGE₂ serinol amide; PGE₂ methyl ester; 16-phenyl tetranor PGE₂; 15(S)-15-methyl PGE₂; 15(R)-15-methyl PGE₂; 8-iso-15-keto PGE₂; 8-iso PGE₂ isopropyl ester; 8-iso-16-cyclohexyltetranor PGE₂; 20-hydroxy PGE₂; 20-ethyl PGE₂; 11-deoxy PGE₁; nocloprost; sulprostone; butaprost; 15-keto PGE₂; 19(R) hydroxy PGE₂; 5-[(1E,3R)-4,4-difluoro-3-hydroxy-4-phenyl-1-buten-1-yl]-1-[6-(2H-tetrazol-5R-yl)hexyl]-2-pyrrolidinone (L-902,688); 2-[3-[(1R,2S,3R)-3-hydroxy-2-[(E,3S)-3-hydroxy-5-[2-(methoxymethyl)phenyl]pent-1-enyl]-5-oxocyclopentyl]sulfanylpropylsulfanyl]acetic acid (ONO-AE1-329); methyl4-[2-[(1R,2R,3R)-3-hydroxy-2-[(E,3S)-3-hydroxy-4-[3-(methoxymethyl)phenyl]but-1-enyl]-5-oxocyclopentyl]ethylsulfanyl]butanoate (ONO-4819); 16-(3-Methoxymethyl)phenyl-\acute{ω}-tetranor-5-thiaPGE₁; 5-{3-[(2S)-2-{(3R)-3-hydroxy-4-[3-(trifluoromethyl)phenyl]butyl}-5-oxopyrrolidin-1-yl]propyl]thiophene-2-carboxylate (PF-04475270); APS-999 Na; [4′-[3-butyl-5-oxo-1-(2-trifluoromethyl-phenyl)-1,5-dihydro-[1,2,4]triazol-4-ylmethyl]-biphenyl-2-sulfonic acid (3-methyl-thiophene-2-carbonyl)-amide]; ((Z)-7-{(1R,4S,5R)-5-[(E)-5-(3-chloro-benzo[b]thiophene-2-yl)-3-hydroxy-pent-1-enyl]-4-hydroxy-3,3-dimethyl-2-oxo-cyclopentyl}-hept-5-enoic acid); Linoleic Acid; 13(s)-HODE; LY171883; Mead Acid; Eicosatrienoic Acid; Epoxyeicosatrienoic Acid; ONO-259; Cay1039; Butaprost; Sulprostone; CAY10399; ONO_8815Ly; ONO-AE1-259; and CP-533,536.

6. The isolated population of T lymphocytes of claim 5, wherein said PGE receptor agonist comprises a compound that selectively binds the PGE₂ EP₂ or PGE₂ EP₄ receptor.

7. The isolated population of T lymphocytes of claim 1, wherein said prostaglandin pathway agonist is selected from the group consisting of PGE₂, dmPGE₂, 15(S)-15-methyl PGE₂, 20-ethyl PGE₂, and 8-iso-16-cyclohexyl-tetranor PGE₂.

8. The isolated population of T lymphocytes of claim 1, wherein said at least one prostaglandin pathway agonist is dmPGE₂.

9. The isolated population of T lymphocytes of claim 1, wherein said glucocorticoid is selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, and ulobetasol.

10. The isolated population of T lymphocytes of claim 1, wherein said at least one glucocorticoid is dexamethasone.

11. The isolated population of T lymphocytes of claim 1, wherein said at least one prostaglandin pathway agonist is dmPGE₂ and said at least one glucocorticoid is dexamethasone.

12. The isolated population of T lymphocytes of claim 1, wherein the concentration of said prostaglandin pathway agonist is about 10 μm.

13. The isolated population of T lymphocytes of claim 1, wherein the concentration of said glucorticoid is about 10 μm.

14. The isolated population of T lymphocytes of claim 1, wherein the T lymphocytes have been contacted with said agent for at least about one hour, about 1 to about 24 hours, about 1 to about 12 hours, about 1 to about 6 hours, about 1 to about 4 hours, or about 2 to about 4 hours.

15. The isolated population of T lymphocytes of claim 1, wherein said isolated population of T lymphocytes has been contacted with said agent at between about 24° C. to about 39° C.

16. The isolated population of T lymphocytes of claim 1, wherein said isolated population of T lymphocytes has been contacted with said agent at about 37° C.

17. The isolated population of T lymphocytes of claim 1, wherein said contacted population of T lymphocytes comprises modulated cells having increased expression of at least one signature gene by at least about two fold compared to the expression in a noncontacted population of T lymphocytes, wherein the at least one signature gene is selected from the group consisting of BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, IL21R, MCAM, SOCS1, TNFSF4, TOB1, TSC22D3, FGL2, NR4A2, AREG, TGFB1, CD55, THFAIP3, and CXCR4.

18. The isolated population of T lymphocytes of claim 1, wherein said contacted population of T lymphocytes comprises modulated cells having increased expression of at least one signature gene by at least about two fold compared to the expression in a noncontacted population of T lymphocytes, wherein the at least one signature gene is selected from the group consisting of ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2.

19. The isolated population of T lymphocytes of claim 17 or claim 18, wherein said signature gene has increased expression by at least 3, 5, or 10 fold compared to the expression in a noncontacted population of T lymphocytes.

20. The isolated population of T lymphocytes of claim 1, wherein the contacted population of T lymphocytes comprises modulated cells:
(i) having an increased level of PD-1;
(ii) having a decreased level of ICOS or 41BB;
(iii) having decreased production of interferon gamma; and/or
(iv) having increased production of interleukin 4 or interleukin 10,
when compared to cells in a noncontacted population of T lymphocytes.

21. The isolated population of T lymphocytes of claim 1, wherein the contacted population of T lymphocytes comprises increased percentage or number of Treg cells, and wherein the Treg cells:
(i) comprise CD4+CD25^{hi}CD127^{lo}Foxp3 cells;
(ii) having an increased level of one or more Treg effector molecules selected from the group consisting of FGL2, NR4A2, AREG, TGFB1, CD55, CCR4, THFAIP3, NLRP3, CTLA4, and CXCR4;
(iii) having improved survival;
(iv) expressing an increased level of genes for GvHD attenuation;
(v) expressing an increased level of genes for protecting against tissue injuries;
and/or
(vi) expressing an increased level of genes for suppressor function.

22. A method of modulating an isolated population of T lymphocytes comprising contacting said isolated population of T lymphocytes ex vivo with a composition comprising at least one prostaglandin pathway agonist, or at least one glucocorticoid, or a combination thereof, wherein said contacted population of T lymphocytes comprises modulated cells
(i) exhibiting a decreased proliferation of CD4+ and/or CD8+ cells;
(ii) exhibiting decreased allogeneic response; and/or
(iii) exhibiting increased persistence when transplanted,
when compared to cells in a noncontacted population of T lymphocytes

23. The method of claim 22, wherein said isolated population of T lymphocytes
(i) comprise peripheral blood mononuclear cells (PBMC), peripheral blood leukocytes (PBL), tumor infiltrating lymphocytes (TIL), or a combination thereof; or
(ii) comprise one or more subpopulations selected from the group consisting of CD4+/CD8+ double positive T cells, CD4+ T cells, CD8+ T cells, CD3+ T cells, naive T cells, effector T cells, cytotoxic T cells, helper T cells, memory T cells, regulator T cells, Th0 cells, Th1 cells, Th2 cells, Th3 (Treg) cells, Th9 cells, Thαβ helper cells, Tfh cells, stem memory T_{SCM} cells, central memory T_{CM} cells, effector memory T_{EM} cells, effector memory T_{EMRA} cells, gamma delta T cells, and any combinations thereof; or
(iii) are derived from peripheral blood, cord blood, or bone marrow; or
(iv) are differentiated in vitro from a stem cell, a definitive hemogenic endothelium, a CD34+ cell, a hematopoietic stem and progenitor cell, a hematopoietic multipotent progenitor cell, or a T cell progenitor cell; or
(v) comprise an exogenous nucleic acid; or
(vi) are human cells.

24. The method of claim 23, wherein said stem cell is an induced pluripotent stem cell (iPSC) or an embryonic stem cell (ESC).

25. The method of claim 23, wherein said exogenous polynucleotide encodes a TCR or a CAR.

26. The method of claim 22, wherein said prostaglandin pathway agonist is
(i) a PGE receptor agonist;
(ii) PGE₂, or a PGE₂ derivative or analogue; or
(iii) a compound selected from the group consisting of PGE₂, prostaglandin I2, 16,16-dimethyl PGE₂ (dmPGE₂); 16,16-dimethyl PGE₂ p-(p-acetamidobenzamido) phenyl ester; 11-deoxy-16,16-dimethyl PGE₂; 9-deoxy-9-methylene-16,16-dimethyl PGE₂; 9-deoxy-9-methylene PGE₂; 9-keto Fluprostenol; 5-trans PGE₂; 17-phenyl-omega-trinor PGE₂; PGE₂ serinol amide; PGE₂ methyl ester; 16-phenyl tetranor PGE₂; 15(S)-15-methyl PGE₂; 15(R)-15-methyl PGE₂; 8-iso-15-keto PGE₂; 8-iso PGE₂ isopropyl ester; 8-iso-16-cyclohexyltetranor PGE₂; 20-hydroxy PGE₂; 20-ethyl PGE₂; 11-deoxy PGE₁; nocloprost; sulprostone; butaprost; 15-keto PGE₂; 19(R) hydroxy PGE₂; 5-[(1E,3R)-4,4-difluoro-3-hydroxy-4-phenyl-1-buten-1-yl]-1-[6-(2H-tetrazol-5R-yl)hexyl]-2-pyrrolidinone (L-902,688); 2-[3-[(1R,2S,3R)-3-hydroxy-2-[(E,3S)-3-hydroxy-5-[2-(methoxymethyl)phenyl]pent-1-enyl]-5-oxocyclopentyl]sulfanylpropylsulfanyl]acetic acid (ONO-AE1-329); methyl4-[2-[(1R,2R,3R)-3-hydroxy-2-[(E,3S)-3-hydroxy-4-[3-(methoxymethyl)phenyl]but-1-enyl]-5-oxocyclopentyl]ethylsulfanyl]butanoate (ONO-4819); 16-(3-Methoxymethyl)phenyl-o-tetranor-5-thiaPGE₁; 5-{3-[(2S)-2-{(3R)-3-hydroxy-4-[3-(trifluoromethyl)phenyl]butyl}-5-oxopyrrolidin-1-yl]propyl]thiophene-2-carboxylate (PF-04475270); APS-999 Na; [4′-[3-butyl-5-oxo-1-(2-trifluoromethyl-phenyl)-1,5-dihydro-[1,2,4]triazol-4-ylmethyl]-biphenyl-2-sulfonic acid (3-methyl-thiophene-2-carbonyl)-amide]; ((Z)-7-{(1R,4S,5R)-5-[(E)-5-(3-chloro-benzo[b]thiophene-2-yl)-3-hydroxy-pent-1-enyl]-4-hydroxy-3,3-dimethyl-2-oxo-cyclopentyl}-hept-5-enoic acid); Linoleic Acid; 13(s)-HODE; LY171883; Mead Acid; Eicosatrienoic Acid; Epoxyeicosatrienoic Acid; ONO-259; Cay1039; Butaprost; Sulprostone; CAY10399; ONO_8815Ly; ONO-AE1-259; and CP-533,536.

27. The method of claim 26, wherein said PGE receptor agonist comprises a compound that selectively binds the PGE₂ EP₂ or PGE₂ EP₄ receptor.

28. The method of claim 22, wherein said prostaglandin pathway agonist is selected from the group consisting of PGE₂, dmPGE₂, 15(S)-15-methyl PGE₂, 20-ethyl PGE₂, and 8-iso-16-cyclohexyl-tetranor PGE₂.

29. The method of claim 22, wherein said prostaglandin pathway agonist is dmPGE₂.

30. The method of claim 22, wherein said glucocorticoid is selected from the group consisting of medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide, or ulobetasol.

31. The method of claim 22, wherein said at least one glucocorticoid is dexamethasone.

32. The method of claim 22, wherein said at least one prostaglandin pathway agonist is dmPGE₂ and said at least one glucocorticoid is dexamethasone.

33. The method of claim 22, wherein the concentration of said prostaglandin pathway agonist is about 10 μm.

34. The method of claim 22, wherein the concentration of said glucocorticoid is about 10 μm.

35. The method of claim 22, wherein the T lymphocytes have been contacted with said agent for at least about one hour, about 1 to about 24 hours, about 1 to about 12 hours, about 1 to about 6 hours, about 1 to about 4 hours, or about 2 to about 4 hours.

36. The method of claim 22, wherein said isolated population of T lymphocytes has been contacted with said agent at between about 24° C. to about 39° C.

37. The method of claim 22, wherein said isolated population of T lymphocytes has been contacted with said agent at about 37° C.

38. The method of claim 22, wherein said contacted population of T lymphocytes comprises modulated cells having increased expression of at least one signature gene by at least about two fold compared to the expression in a noncontacted population of T lymphocytes, wherein the at least one signature gene is selected from the group consisting of BCL2L11, FOS, FOSL2, MCL1, NLRP3, NR4A3, SGK1, VEGFA, CCNA1, CCND1, CCND3, CORO1C, HBEGF, S100P, CCR4, CXCL2, CXCL3, CXCL5, CXCL6, CXCR2, CEBPB, CEBPD, CTLA4, FGL2, FKBP5, ICOSLG, IL21R, MCAM, SOCS1, TNFSF4, TOB1, TSC22D3, FGL2, NR4A2, AREG, TGFB1, CD55, THFAIP3, and CXCR4.

39. The method of claim 22, wherein said contacted population of T lymphocytes comprises modulated cells having increased expression of at least one signature gene by at least about two fold compared to a non-contacted population of T lymphocytes, said one or more signature genes being selected from the group consisting of ATF3, CREM, GEM, CXCL2, MMP9, PLAUR, AREG, BCL2A1, DUSP4, FOS, FOSL2, JUN, MYC, NR4A2, NR4A3, SOCS1, SOCS3, and ULBP2.

40. The method of claim 38 or claim 39, wherein said signature gene has increased expression by at least 3, 5, or 10 fold compared to a noncontacted population of T lymphocytes.

41. The method of claim 22, wherein the contacted population of T lymphocytes comprises modulated cells
(i) having an increased level of PD-1;
(ii) having a decreased level of ICOS or 41BB;
(iii) having decreased production of interferon gamma; and/or
(iv) having increased production of interleukin 4 or interleukin 10,
when compared to cells in a noncontacted population of T lymphocytes.

42. The method of claim 22, wherein the contacted population of T lymphocytes comprises increased percentage or number of Treg cells, and wherein the Treg cells:
(i) comprise CD4+CD25^{hi}CD127^{lo}Foxp3⁺ cells;
(ii) having an increased level of one or more Treg effector molecules selected from the group consisting of FGL2, NR4A2, AREG, TGFB1, CD55, CCR4, THFAIP3, NLRP3, CTLA4, and CXCR4;
(iii) having improved survival;
(iv) expressing an increased level of genes for GvHD attenuation;
(v) expressing an increased level of genes for protecting against tissue injuries; and/or
(vi) expressing an increased level of genes for suppressor function.

43. The method of claim 22, further comprising washing said contacted population of T lymphocytes with a buffer substantially free of said agent.

44. The method of claim 22, further comprising administering said contacted population of T lymphocytes to a subject in need of cell therapy.

45. The method of claim 44, wherein said subject
(i) is a candidate for bone marrow or stem cell transplantation, or said subject has received chemotherapy or irradiation therapy;
(ii) has received bone marrow ablative or non-myeolablative chemotherapy or radiation therapy;
(iii) has a hyperproliferative disorder or a cancer of hematopoietic system; or
(iv) has a solid tumor; or
(v) has a virus infection or a disease associated with virus infection.

46. The method of claim 45, wherein said hyperproliferative disorder or cancer of hematopoietic system is leukemia, lymphoma, or myeloma.

47. The method of claim 45, wherein said solid tumor is breast cancer, ovarian cancer, brain cancer, prostate cancer, lung cancer, colon cancer, skin cancer, liver cancer, pancreatic cancer, or sarcoma.

48. The method of claim 45, wherein said virus infection is associated with HIV (human immunodeficiency virus), RSV (Respiratory Syncytial Virus), EBV (Epstein-Barr virus), CMV (cytomegalovirus), adenovirus, or BK polyomavirus.

49. A pharmaceutical composition comprising the contacted population or subpopulation of T lymphocytes of claim 1 and a pharmaceutically acceptable medium.

50. The pharmaceutical composition of claim 49, wherein said composition is substantially free of prostaglandin pathway agonist and glucocorticoid.

51. A method of treating a subject in need of cell therapy comprising administering the contacted population or subpopulation of T lymphocytes of claim 1 or the pharmaceutical composition of claim 47 to said subject.

52. The method of claim 51, wherein said subject
(i) is a candidate for bone marrow or stem cell transplantation, or the subject has received chemotherapy or irradiation therapy;
(ii) has received bone marrow ablative or non-myeolablative chemotherapy or radiation therapy;
(iii) has a hyperproliferative disorder or a cancer of hematopoietic system;
(iv) has a solid tumor; or
(v) has a virus infection or a disease associated with virus infection.

53. The method of claim 52, wherein said hyperproliferative disorder or cancer of hematopoietic system is leukemia, lymphoma, or myeloma.

54. The method of claim 52, wherein said solid tumor is breast cancer, ovarian cancer, brain cancer, prostate cancer, lung cancer, colon cancer, skin cancer, liver cancer, pancreatic cancer, or sarcoma.

55. The method of claim 52, wherein said virus infection is associated with HIV (human immunodeficiency virus), RSV (Respiratory Syncytial Virus), EBV (Epstein-Barr virus), CMV (cytomegalovirus), adenovirus, or BK polyomavirus.
